(19) United States
(12) Patent Application Publication
(10) Pub. No.: US 2025/0368978 A1
(43) Pub. Date: Dec. 4, 2025

(54) SQUALENE HOPENE CYCLASE VARIANTS FOR PRODUCING SCLAREOLIDE

(71) Applicant: INTERNATIONAL FLAVORS & FRAGRANCES INC., NEW YORK, NY (US)

(72) Inventors: Lyndsey Jane BANNON, Belfast (IE); Daniel Fernando Andrade Ribeiro DOURADO, Vila do Conde (PT); Stefan MIX, Belfast (IE); Thomas Shaw MOODY, Craigavon (GB); Derek John Quinn, Craigavon (GB); Paul D. JONES, Aberdeen, NJ (US); Anubhav P.S. NARULA, Hazlet, NJ (US); John P. CHERKAUSKAS, Jr., Burlington, NJ (US)

(21) Appl. No.: 18/874,932

(22) PCT Filed: Jun. 14, 2023
(86) PCT No.: PCT/US2023/068411

§ 371 Date: Dec. 13, 2024

Related U.S. Application Data
(60) Provisional application No. 63/352,352, filed on Jun. 15, 2022.

(51) Int. Cl. C12N 9/90; C12P 17/04

(52) U.S. Cl. CPC C12N 9/90; C12P 17/04; C12Y 504/99017

(57) ABSTRACT

Variants of squalene hopene cyclase (SHC) are provided for enzymatically converting homofarnesoic acid to sclareolide, which can be non-enzymatically converted to ambrox.

### CROSS-REFERENCE TO RELATED APPLICATION

This application claims priority from U.S. provisional application No. 63/352352, filed Jun. 15, 2022, the contents of which are hereby incorporated by reference in their entirety.

### INCORPORATION BY REFERENCE OF THE SEQUENCE LISTING

The present application is being filed with a Sequence Listing in electronic format. The Sequence Listing is provided as a file entitled IFF652WOPCT_SequenceListing.xml, created on Jun. 13, 2023 which is 95 bytes in size. The information in the electronic format of the Sequence Listing is incorporated by reference in its entirety.

### TECHNICAL FIELD

Variants of squalene hopene cyclase (SHC) are provided for enzymatically converting homofarnesoic acid to sclareolide, which can be non-enzymatically converted to ambrox.

### BACKGROUND

Compounds with the dodecahydronaphtho [2,1-b]furan skeleton are of great economic importance as aroma chemicals. Among these, (3aR,5aS,9aS,9bR)-dodecahydro-3a,6,6,9a-tetramethylnaphtho [2,1-b]furan), known as ambrox, is of particular importance for providing base notes of perfume compositions. Originally obtained from sperm whales' ambergris, synthetic methods have been developed for the production of ambrox. In one approach, sclareol, a constituent of clary sage (Salvia sclarea), is used as a starting material. Oxidative degradation of sclareol with, e.g., chromic acid, permanganate, H₂O₂ or ozone provides sclareolide, which is subsequently reduced, e.g., using LiAlH₄ or NaBH₄ to give ambrox-1,4-diol. Alternatively, sclareolide can be prepared from sclareol by means of a biotransformation using Hyphozyma roseoniger (EP 0204009). Finally, ambra diol or tetranor labdane diol is cyclized in a series of chemical processes to give compound ambrox ((−)-2). The preparation of the racemate of ambrox, rac-2, has been accomplished, inter alia, via homofarnesylic acid and 4-(2,6,6-trimethylcyclohex-1-enyl)butan-2-one.

In another approach, ambrox is biocatalytically prepared using squalene hopene cyclase (SHC; Scheme 1) (Neumann, et al. (1986) Biol. Chem. Hoppe Seyler 367:723).

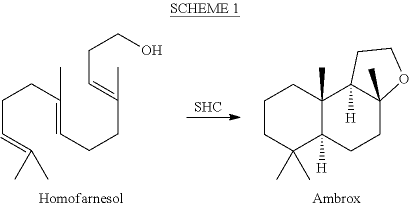

While SHC naturally catalyzes the cyclization of squalene to hopane, catalysis of ambrox is a secondary reaction with a specific activity of 0.02 mU/mg protein. SHC from Alicyclobacillus acidocaldarius (formerly Bacillus acidocaldarius), Zymomonas mobilis and Bradyrhizobium japonicum have been purified and characterized in terms of their natural (e.g., squalene) and non-natural substrates (e.g., homofarnesol and citral). See, e.g., WO 2010/139719, WO 2012/066059, JP 2009060799 and Seitz, et al. (2012) J. Molecular Catalysis B: Enzymatic 84:72-77). In addition, WO 2016/170099 describes SHC variants with improved rates of conversion of E,E-homofarnesol to ambrox. US11091752 provides further SHC variants.

### SUMMARY

Described are variant SHC molecules and methods of use, thereof, for producing sclareolide from homofarnesoic acid, which sclareolide can be non-enzymatically converted to ambrox.

In an aspect is provided a variant Squalene Hopene Cyclase (SHC) polypeptide having at least 60% sequence identity to SEQ ID NO: 2 and including an amino acid substitution at one or more positions corresponding to positions V45, Q54, Q178, M184, V222, R249, I278, Y284, T326, R348, A574, A683, relative to SEQ ID NO: 2, wherein the variant demonstrates increased sclareolide production compared to SEQ ID NO: 2. In some embodiments, the amino acid substitution at the one or more positions is V45L, Q54E, Q178S, M184T, V222Q, V222R, V222K, R249R, I278T, I278A, I278S, Y284W, T326S, R348R, A574A, A683E, or any combination thereof. In some embodiments, the variant SHC polypeptide includes amino acid substitutions: (a) V45L, Q54E, 1278T, T326S; (b) V45L, Q54E, M184T, R249R, I278T, T326S; (c) V45L, Q54E, V222Q, R249R, I278T, T326S; (d) V45L, Q54E, R249R, 1278T, T326S; (c) V45L, Q54E, V222R, 1278T, T326S; (f) V45L, Q54E, V222K, 1278T, T326S; (g) V45L, Q54E, V222K, 1278S, T326S; (h) V45L, Q54E, V222K, 1278A, T326S; (i) V45L, Q54E, R249R, 1278A, T326S; (j) V45L, Q54E, R249R, 1278T, T326S, A574A; (k) V45L, Q54E, V222K, R249R, 1278T, T326S; (1) V45L, Q54E, Q178S, V222K, I278S, T326S; (m) V45L, Q54E, Q178S, V222R, 1278T, T326S; (n) V45L, Q54E, V222Q, R249R, I278T, T326S, A683E; (0) V45L, Q54E, V222Q, Y284W, R249R, 1278T, T326S; (p) V45L, Q54E, V222R, 1278T, T326S, R348R; (q) V45L, Q54E, V222K, R249R, I278A, T326S; or (r) V45L, Q54E, Q178S, V222K, 1278T, T326S. In some embodiments, the variant SHC polypeptide has at least 70%, 80%, 90%, 95%, 99%, or 100% sequence identity to the sequence set forth by SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

In an aspect is provided a nucleic acid molecule comprising a nucleic acid sequence encoding a variant SHC polypeptide described herein. In some embodiments, the nucleic acid sequence: i) encodes an amino acid sequence having at least 70%, 80%, 90%, 95%, 99%, or 100% sequence identity to the sequence set forth by SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25; ii) has at least 70%, 80%, 90%, 95%, 99%, or 100% sequence identity to the sequence set forth by SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42; or iii) hybridizes under stringent conditions to a nucleic acid sequence having a sequence complementary to the sequence set forth by SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42. In some embodiments, the nucleic acid sequence includes a heterologous regulatory sequence, optionally a promoter sequence.

In an aspect is provided an expression vector including a nucleic acid molecule encoding a variant SHC described herein. In an aspect is an expression vector including a nucleic acid molecule described herein. In an aspect is provided a recombinant host cell including a nucleic acid molecule encoding a variant SHC described herein. In an aspect is provided a recombinant host cell including an expression vector described herein.

In an aspect is provided a method for producing sclareolide including contacting homofarnesoic acid with a variant SHC polypeptide described herein or a recombinant host cell described herein. In some embodiments, the method includes collecting the sclareolide. In some embodiments, the homofarnesoic acid comprises (3E,7E) homofarnesoic acid. In some embodiments, the method includes non-enzymatically converting the sclareolide to ambrox.

Aspects and embodiments of the variant molecules and methods are described in the following, independently numbered paragraphs.
1. In one aspect, a non-naturally occurring variant of a wild-type squalene hopene cyclase (SHC) polypeptide is described, having at least 80% sequence identity to SEQ ID NO: 1 and comprising first amino acid substitutions, relative to SEQ ID NO: 1, at positions V45, Q54, I278 and T326, and a second amino acid substitution, relative to SEQ ID NO: 2, at position M184 and/or V222, wherein the variant demonstrates increased sclareolide production compared to a variant of the wild-type SHC having only the amino acid substitutions V45L, Q54E, I278T and T326S, relative to SEQ ID NO: 1 for numbering.
2. In some embodiments of the variant SHC of paragraph 1, the first amino acid substitutions are V45L, Q54E, I278T and T326S.
3. In some embodiments of the variant SHC of paragraph 1 or 2, the second amino acid substitution is selected from the group consisting of M184T, V222Q, V222R, and V222K, or combinations, thereof.
4. In some embodiments, the variant SHC of paragraph 1 has amino acid substitutions selected from the group consisting of: (i) V45L, Q54E, M184T, I278T and T326S, (ii) V45L, Q54E, V222Q, I278T and T326S, (iii) V45L, Q54E, I278T and T326S, (iv) V45L, Q54E, V222R, I278T and T326S, (v) V45L, Q54E, V222K, I278T and T326S, or (vi) V45L, Q54E, V222K, 1278S and T326S, all relative to SEQ ID NO: 1 for numbering, wherein the variant demonstrates increased sclareolide production compared to a variant of the wild-type SHC having only the amino acid substitutions V45L, Q54E, I278T and T326S, relative to SEQ ID NO: 2 for numbering.
5. In some embodiments, the variant SHC of any of paragraphs 1-4 has at least 90% amino acid sequence identity to SEQ ID NO: 2.
6. In another aspect, a recombinant vector encoding the variant SHC polypeptide of any of paragraphs 1-5 is provided.
7. In another aspect, a recombinant host cell comprising the recombinant vector of paragraph 6 is provided.
8. In another aspect, a method for producing sclareolide comprising (a) contacting homofarnesoic acid with (i) the variant of the wild-type SHC having only the amino acid substitutions V45L, Q54E, I278T and T326S, relative to SEQ ID NO: 1, or (ii) the variant of the wild-type SHC of any of paragraphs 1-5; and (b) collecting sclareolide thereby produced, is provided.
9. In another aspect, a method for producing sclareolide comprising (a) contacting homofarnesoic acid with the recombinant host cell of paragraph 7, and (b) collecting sclareolide thereby produced, is provided.
10. In some embodiments of the method of paragraph 8 or 9, the homofarnesoic acid comprises (3E,7E) homofarnesoic acid.
11. In some embodiments, the method of any of paragraphs 8-10 further comprises non-enzymatically converting the sclareolide to ambroxen.

Each of the aspects and embodiments described herein are capable of being used together, unless excluded either explicitly or clearly from the context of the embodiment or aspect.

These and other aspect and embodiments of the variant molecules and methods are described below, with reference to any appended Drawings.

### BRIEF DESCRIPTION OF THE DRAWINGS

FIG. 1 provides an amino acid sequence comparison of Gluconobacter morbifer squalene hopene cyclase (GmSHC) with related SHC enzymes from Z. mobili s(ZmSHC), Bradyrhizobium sp. (BspSHC), Rhodopseudomonas palustris (RpSHC), Streptomyces coelicolor (ScSHC), Burkholderia ambifaria (BamSHC), Bacillus anthracis (BanSHC) and A. acidocaldarius (AaSHC). Underlined residues represent the core sequence Gln-Xaa-Xaa-Xaa-Gly-Xaa-Trp (SEQ ID NO: 3) and bolded residues represent the Asp-Xaa-Asp-Asp-Thr-Ala (SEQ ID NO: 4) active site motif.

### DETAILED DESCRIPTION

### 1. Definitions and Abbreviations

Prior to describing the variants and methods in detail, the following terms are defined. Unless defined otherwise, all technical and scientific terms used herein have the same meaning as commonly understood by one of ordinary skill in the art. The singular forms “a,” “an,” and “the” include plural referents unless the context clearly dictates otherwise. The present document is organized into a number of sections for ease of reading; however, the reader will appreciate that statements made in one section may apply to other sections. In this manner, the headings used for different sections of the disclosure should not be construed as limiting.

All publications, including patent documents, scientific articles, and databases, referred to in this application are incorporated by reference in their entirety for all purposes to the same extent as if each individual publication were individually incorporated by reference. Nothing herein is to be construed as an admission that such publications constitute prior art to the claims appended hereto. If a definition set forth herein is contrary to or otherwise inconsistent with a definition set forth in the patents, applications, published applications, and other publications that are herein incorporated by reference, the definition set forth herein prevails over the definition that is incorporated herein by reference.

### 1.1. Definitions

As used herein, the term “ambrox” refers to (3aR,5aS,9aS,9bR)-dodecahydro-3a,6,6,9a-tetramethylnaphtho [2,1-b]furan), which is known commercially as AMBROX (Firmenich), Ambroxan (Henkel) AMBROFIX® (Givaudan), AMBERLYN® (Quest), CETALOX® Laevo (Firmenich), AMBERMOR® (International Flavors and Fragrances, and AROMOR® and/or norambrenolide Ether (Pacific). The desirable sensory benefits of ambrox come from the (−) stereoisomer rather than the (+) enantiomer. The odor of the (−) stereoisomer is described as musk-like, woody, warm or ambery whereas the (+) enantiomer has a relatively weak odor note.

As used herein, “GmSHC” refers to the squalene hopene cyclase isolated from Gluconobacter morbifer. In particular, when not modified by “variant,” “GmSHC” refers to a wild-type protein having the amino acid sequence according to SEQ ID NO: 1. By comparison, “variant GmSHC” or “GmSHC variant” refers to a GmSHC in which the amino acid sequence is altered compared to the amino acid sequence of the reference (or wild-type) GmSHC sequence of SEQ ID NO: 1. In some embodiments, when not modified by “variant,” “GmSHC” refers to a wild-type protein having the amino acid sequence according to SEQ ID NO: 2. In some embodiments, “variant GmSHC” or “GmSHC variant” refers to a GmSHC in which the amino acid sequence is altered compared to the amino acid sequence of the reference (or wild-type) GmSHC sequence of SEQ ID NO: 2. The terms variant SHC and SHC variant may be used alternatively herein to refer to variant GmSHC and GmSHC variant.

As used herein, the term “target yield” refers to the gram of recoverable product per gram of feedstock (which can be calculated as a percent molar conversion rate).

As used herein, the term “activity” means the ability of an enzyme to react with a substrate to provide a target product. The activity can be determined in what is known as an activity test via the increase of the target product, the decrease of the substrate (or starting materials) or via a combination of these parameters as a function of time.

As used herein, the term “target productivity” refers to the amount of recoverable target product in grams per liter of fermentation capacity per hour of bioconversion time (i.e., time after the substrate was added). Moreover, a GmSHC variant can exhibit a modified target yield factor compared to the reference GmSHC protein.

As used herein, the term “target yield factor” refers to the ratio between the product concentration obtained and the concentration of the GmSHC variant (for example, purified GmSHC enzyme or an extract from the recombinant host cells expressing the GmSHC enzyme) in the reaction medium.

As used herein, the term “recombinant host,” also referred to as a “genetically modified host cell” or “transgenic cell” denotes a host cell that includes a heterologous nucleic acid or the genome of which has been augmented by at least one incorporated DNA sequence.

As used herein, the term “nucleic acid molecule,” refers to polynucleotides of the disclosure which can be DNA, cDNA, genomic DNA, synthetic DNA, or RNA, and can be double-stranded or single-stranded, the sense and/or an antisense strand.

As used herein, the term “isolated DNA,” as used herein, refers to nucleic acids or polynucleotides isolated from a natural source (e.g., Gluconobacter morbifer) or nucleic acids or polynucleotides produced by recombinant DNA techniques, e.g., a DNA construct include a polynucleotide heterologous to a host cell, which is optionally incorporated into the host cell.

“Hybridization” and grammatical variants thereof refer to the process by which one strand of nucleic acid forms a duplex with, i.c., base pairs with, a complementary strand, as occurs during blot hybridization techniques and PCR techniques. Stringent hybridization conditions are exemplified by hybridization under the following conditions: 65° C. and 0.1X SSC (where 1X SSC=0.15 M NaCl, 0.015 M Na₃ citrate, pH 7.0). Hybridized, duplex nucleic acids are characterized by a melting temperature (Tm), where one half of the hybridized nucleic acids are unpaired with the complementary strand. Mismatched nucleotides within the duplex lower the Tm.

As used, herein, an “expression vector” includes a recombinant nucleic acid molecule encoding wild-type GmSHC or a SHC variant or homolog, as described herein and the necessary regulatory regions suitable for expressing the polypeptide. The choice of expression vector, e.g. plasmid, cosmid, virus or phage vector, will often depend on the host cell into which it is to be introduced. In some embodiments, the vector is a plasmid. In some embodiments, the recombinant nucleic acid molecule encoding wild-type GmSHC or a SHC variant or homolog, as described herein, is operably linked to various promoters and regulators to drive expression when present, for example, in a host cell. An expression vector may also be referred to herein as a recombinant vector.

As used herein, the term “transformed” refers to the introduction of an exogenous or heterologous DNA into a cell. The transforming DNA may or may not be integrated, i.e., covalently linked into the genome of the cell.

As used herein, the term “selective crystallization” refers to a process step whereby (−)-sclareolide is caused to crystallize from a solvent while the remaining isomers remain dissolved in the crystallizing solvent.

In certain embodiments, the final product is isolated (−)-sclareolide. The term “isolated” as used with reference to (−)-sclareolide, refers to a bioconversion product that has been separated or purified from components which accompany it. An entity that is produced in a cellular system different from the source from which it naturally originates is “isolated” because it will necessarily be free of components which naturally accompany it. The degree of isolation or purity can be measured by any appropriate method, e.g., gas chromatography (GC), HPLC or NMR analysis.

### 1.2 Abbreviations and acronyms

The following abbreviations/acronyms have the following meanings unless otherwise specified:
° C. degrees Centigrade
dH₂O or DI deionized water
g or gm grams
FID flame ionization detector
GC gas chromatography
hr(s) hour/hours
kg kilograms
LGA Lamarckian genetic algorithm
M molar
mg milligrams
min(s) minute/minutes
mL and ml milliliters
mm millimeters
mM millimolar
MW molecular weight
SDS sodium dodecyl sulfate
PAGE polyacrylamide gel electrophoresis
sec seconds
U units
v/v volume/volume
w/v weight/volume
w/w weight/weight
wt % weight percent

### 2. Variants Squalene Hopene Cyclase Enzymes with Altered Product Profiles

Described are variants of a parental squalene hopene cyclase (SHC), exemplified by a homofarnesol-ambrox cyclase (HAC) isolated from Gluconobacter morbifer, that demonstrate improved conversion of homofarnesoic acid (HFA) to sclareolide compared to the relative parental enzymes. The direct enzymatic conversion of HFA to ambrox has been described but is not an efficient process. The enzymatic conversion of HFA to sclareolide, followed by the biochemical conversion of sclareolide to ambrox, may be more efficient and less expensive than the direct enzymatic conversion. Variants demonstrating improved conversion of HFA to sclareolide have not heretofore been described.

The amino acid sequence of wild-type Gluconobacter morbifer SHC (i.e., (GmSHC)) is available under GENBANK Accession Nos. WP_040507485 and EHH69691. The amino acid sequence of wild-type GmSHC is provided, below, as SEQ ID NO: 1:

MLPEAVSSAC DWLIDQQKPD GHWVGPVESN ACMEAQWCLA
LWELGQEDHP LRPRLAQALL EMQREDGSWG IYVGADHGDI
NITVEAYAAL RSMGYAADMP IMAKSAAWIQ QKGGLRNVRV
FTRYWLALIG EWPWDKIPNL PPEIIWLPDN FIFSIYNFAQ
WARAIMMPLT ILSARRPSRP LLPENRLDGL FPEGRENFDY
ELPVKGEEDL WGRFFRAADK GLHSLQSFPV RRFVPREAAI
RHVIEWIIRH QDADGGWGGI QPPWIYGLMA LSVEGYPLHH
PVLAKAMDAL NDPGWRRDKG DASWIQAINS PVWDIMLAVL
ALHDAGAEDR YSPQMDKAIG WLLDRQVRVK GDWSIKLPDT
EPGGWAFEYA NDKYPDTDDT AVALIALAGC RHRPEWRERD
IEGAISRGVN WLLAMQSSSG GWGAFDKDNN RSILTKIPFC
DFGEALDPPS VDVTAHVLEA FGLLGISRNH PSVQKALAYI
RSEQERNGAW FGRWGVNYVY GTGAVLPALA AIGEDMTQPY
IVRACDWLMS VQQENGGWGE SCASYMDINA VGHGVATASQ
TAWALIGLLA AKRPKDREAI ARGCQFLIER QEDGSWTEEE
YTGTGFPGYG VGQAIKLDDP SLPDRLLQGA ELSRAFMLRY
DLYRQYFPVM ALSRARRMMK EDASAAA

An amino acid sequence of wild-type GmSHC is also provided in SEQ ID NO: 2:

MSPADISTKS SSFQRLDNML PEAVSSACDW LIDQQKPDGH
WVGPVESNAC MEAQWCLALW FLGQEDHPLR PRLAQALLEM
QREDGSWGIY VGADHGDINT TVEAYAALRS MGYAADMPIM
AKSAAWIQQK GGLRNVRVFT RYWLALIGEW PWDKIPNLPP
EIIWLPDNFI FSIYNFAQWA RATMMPLTIL SARRPSRPLL
PENRLDGLFP EGRENFDYEL PVKGEEDLWG RFFRAADKGL
HSLQSFPVRR FVPREAAIRH VIEWIIRHQD ADGGWGGIQP
PWIYGLMALS VEGYPLHHPV LAKAMDALND PGWRRDKGDA
SWIQATNSPV WDTMLAVLAL HDAGAEDRYS PQMDKAIGWL
LDRQVRVKGD WSIKLPDTEP GGWAFEYAND KYPDTDDTAV
ALIALAGCRH RPEWRERDIE GAISRGVNWL LAMQSSSGGW
GAFDKDNNRS ILTKIPFCDF GEALDPPSVD VTAHVLEAFG
LLGISRNHPS VQKALAYIRS EQERNGAWFG RWGVNYVYGT
GAVLPALAAI GEDMTQPYIV RACDWLMSVQ QENGGWGESC
ASYMDINAVG HGVATASQTA WALIGLLAAK RPKDREAIAR
GCQFLIERQE DGSWTEEEYT GIGFPGYGVG QAIKLDDPSL
PDRLLQGAEL SRAFMLRYDL YRQYFPVMAL SRARRMMKED
ASAAA

An alignment of the GmSHC amino acid sequence with similar SHC amino acid sequences from Z. mobilis, Bradyrhizobium sp., R. palustris, S. coelicolor, B. ambifaria, B. anthracis and A. acidocaldarius indicates amino acid sequence identities ranging between 37% and 76% (Table 1). Table 1 shows alignments with SEQ ID NO: 1.

TABLE 1
Alignment of various SHC amino acid
sequences and identity with GmSHC.

| Source organism | Accession No. | Identity (%) |
|---|---|---|
| Zymomonas mobilis | Q5NM88 | 76 |
| Bradyrhizobium sp. | A5EBP6 | 72 |
| Burkholderia ambifaria | Q0B5S3 | 37 |
| Bacillus. anthracis | A0A0E0W268 | 48 |
| Rhodopseudomonas palustris | WP_011665849 | 66 |
| Streptomyces coelicolor | Q9X7V9 | 45 |
| Alicyclobacillus acidocaldarius | P33247 | 44 |

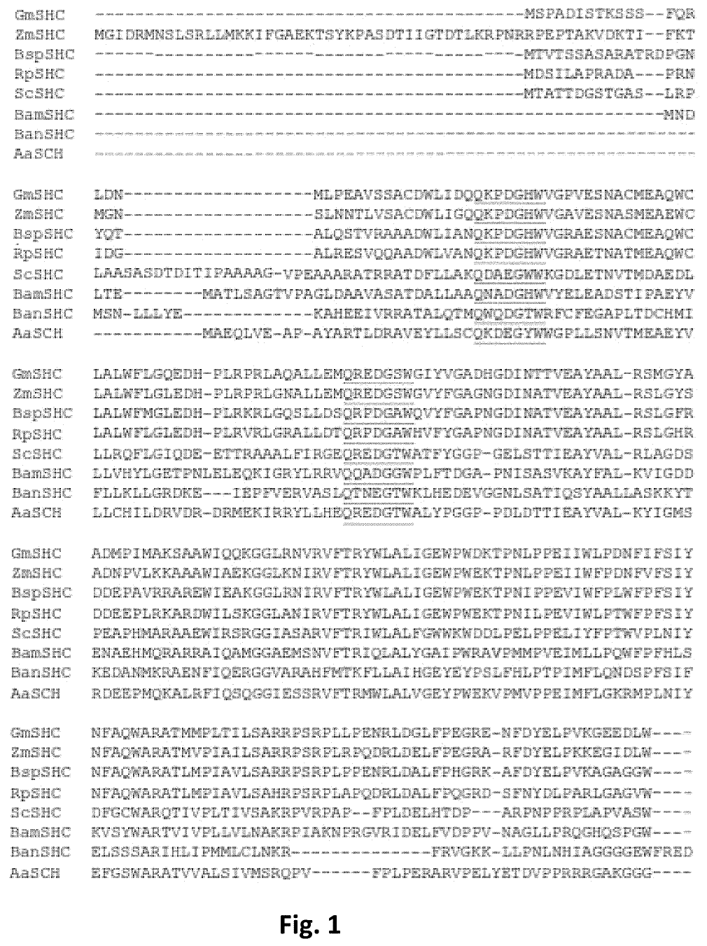

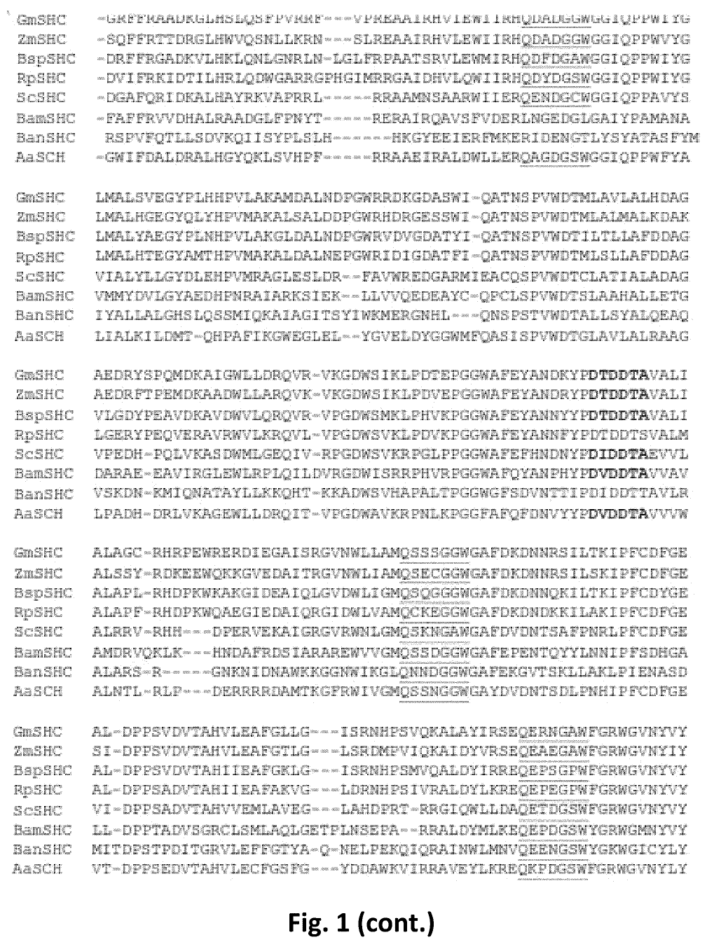

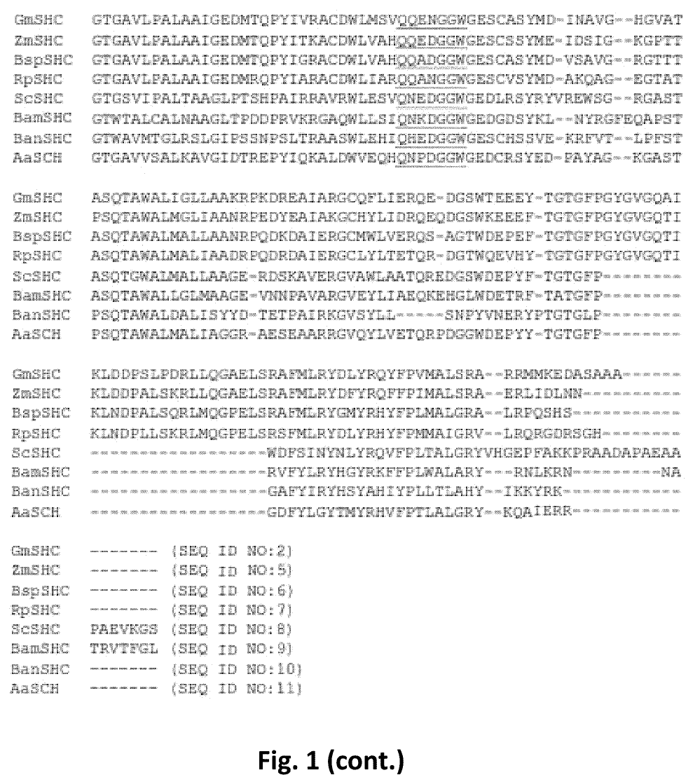

SHC contain the core sequence Gln-Xaa-Xaa-Xaa-Gly-Xaa-Trp (SEQ ID NO: 3) (Reipen et al. (1995) Microbiology 141:155-61), as well as the Asp-Xaa-Asp-Asp-Thr-Ala (SEQ ID NO: 4) motif, which correlates with the SHC active site (Wendt et al. (1997) Science 277:1811-5). (See FIG. 1). The data presented herein demonstrate that variants or variants of the SHC enzyme, when expressed in a heterologous host cell, e.g., E. coli, can readily convert HFA to sclareolide, which can then be converted non-enzymatically to ambrox.

As used herein, the term “amino acid alteration” means an insertion of one or more amino acid residues, a deletion of one or more amino acid residues or a substitution (which may be conservative, non-conservative or synonymous) of one or more amino acid residues relative to the amino acid sequence of a reference amino acid sequence (such as, for example, the wild-type amino acid sequence of SEQ ID NO:2). The amino acid alteration can be easily identified by a comparison of the amino acid sequences of the GmSHC derivative amino acid sequence with the amino acid sequence of the reference or wild-type GmSHC.

Conservative amino acid substitutions may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic-Met, Ala, Val, Leu, Ile; (2) neutral hydrophilic-Cys, Ser, Thr, Asn, Gln; (3) acidic-Asp, Glu; (4) basic-His, Lys, Arg; (5) residues that influence chain orientation-Gly, Pro; and (6) aromatic: Trp, Tyr, Phe. Accordingly, as used herein, the term “conservative substitutions” means an exchange of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt alpha-helices. Some preferred conservative substitutions within the above six groups are exchanges within the following sub-groups: (i) Ala, Val, Leu and Ile; (ii) Ser and Thr; (iii) Asn and Gln: (iv) Lys and Arg; and (v) Tyr and Phe. Given the known genetic code, and recombinant and synthetic DNA techniques, the skilled scientist readily can construct DNAs encoding the conservative amino acid variants. Synonymous or silent mutations, although not altering the amino acid sequence of the encoded protein directly, can still influence splicing accuracy or efficiency. A silent or synonymous mutation may be referred to herein as a substitution.

As used herein, “non-conservative substitutions” or “non-conservative amino acid exchanges” are defined as exchanges of an amino acid by another amino acid listed in a different group of the six standard amino acid groups (1) to (6) as shown above. Typically, the GmSHC derivatives of the present disclosure are prepared using non-conservative substitutions that alter the biological function of the wild-type GmSHC.

Amino acid substitutions may be introduced using known protocols of recombinant gene technology including PCR, gene cloning, site-directed mutagenesis of cDNA, transformation of host cells, and in vitro transcription, which may be used to introduce such changes to the SHC sequence resulting in a SHC variant enzyme. The variants can then be screened for SHC functional activity.

The SHC variant may have from about 1 to about 45 amino acid substitutions, about 1 to about 40 amino acid substitutions, about 1 to about 35 amino acid substitutions, about 1 to about 30 amino acid substitutions, about 1 to about 25 amino acid substitutions, from about 1 to about 20 amino acid substitutions, about 1 to about 15 amino acid substitutions, about 1 to about 10 amino acid substitutions, or from about 1 to about 5 amino acid substitutions relative to the amino acid sequence of the reference (or wild-type) SHC sequence according to SEQ ID NO: 1.

The SHC variant may have from about 1 to about 45 amino acid substitutions, about 1 to about 40 amino acid substitutions, about 1 to about 35 amino acid substitutions, about 1 to about 30 amino acid substitutions, about 1 to about 25 amino acid substitutions, from about 1 to about 20 amino acid substitutions, about 1 to about 15 amino acid substitutions, about 1 to about 10 amino acid substitutions, or from about 1 to about 5 amino acid substitutions relative to the amino acid sequence of the reference (or wild-type) SHC sequence according to SEQ ID NO: 2.

Alternatively, the SHC variant can have at least 5, at least 10 amino acid, or at least 15 amino acid substitutions relative to the amino acid sequence of the reference (i.e. wild-type) SHC sequence according to SEQ ID NO: 1, but ideally not more than about 30 or 40 amino acid substitutions. In various embodiments, the SHC variant may have about 1 amino acid substitution, about 2 amino acid substitutions, about 3 amino acid substitutions, about 4 amino acid substitutions, about 5 amino acid substitutions, about 6 amino acid substitutions, about 7 amino acid substitutions, about 8 amino acid substitutions, about 9 amino acid substitutions, about 10 amino acid substitutions, about 11 amino acid substitutions, about 12 amino acid substitutions, about 15 amino acid substitutions, about 20 amino acid substitutions, about 25 amino acid substitutions, about 30 amino acid substitutions, about 35 amino acid substitutions, about 40 amino acid substitutions, about 45 amino acid substitutions, or about 50 amino acid substitutions relative to the reference SHC.

In some embodiments, the SHC variant can have at least 5, at least 10 amino acid, or at least 15 amino acid substitutions relative to the amino acid sequence of the reference (i.e. wild-type) SHC sequence according to SEQ ID NO: 2, but ideally not more than about 30 or 40 amino acid substitutions. In various embodiments, the SHC variant may have about 1 amino acid substitution, about 2 amino acid substitutions, about 3 amino acid substitutions, about 4 amino acid substitutions, about 5 amino acid substitutions, about 6 amino acid substitutions, about 7 amino acid substitutions, about 8 amino acid substitutions, about 9 amino acid substitutions, about 10 amino acid substitutions, about 11 amino acid substitutions, about 12 amino acid substitutions, about 15 amino acid substitutions, about 20 amino acid substitutions, about 25 amino acid substitutions, about 30 amino acid substitutions, about 35 amino acid substitutions, about 40 amino acid substitutions, about 45 amino acid substitutions, or about 50 amino acid substitutions relative to the reference SHC.

In some embodiments, the amino acid substitution is a silent mutation. Any one or more of the substitutions described herein may be a silent mutation.

In these or other aspects, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the reference SHC of SEQ ID NO: 1.

In some aspects, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the reference SHC of SEQ ID NO: 2.

In some embodiments, the variant SHC polypeptide includes an amino acid substitution at one or more positions corresponding to positions V45, Q54, Q178, M184, V222, R249, I278, Y284, T326, R348, A574, A683, relative to SEQ ID NO: 2. In some embodiments, the variant SHC polypeptide includes an amino acid substitution at 2 or more positions corresponding to positions V45, Q54, Q178, M184, V222, R249, I278, Y284, T326, R348, A574, A683 relative to SEQ ID NO: 2. In some embodiments, the variant SHC polypeptide includes an amino acid substitution at 3 or more positions corresponding to positions V45, Q54, Q178, M184, V222, R249, I278, Y284, T326, R348, A574, A683, relative to SEQ ID NO: 2. In some embodiments, the variant SHC polypeptide includes an amino acid substitution at 4 or more positions corresponding to positions V45, Q54, Q178, M184, V222, R249, I278, Y284, T326, R348, A574, A683, relative to SEQ ID NO: 2. In some embodiments, the variant SHC polypeptide includes an amino acid substitution at 5 or more positions corresponding to positions V45, Q54,Q178, M184, V222, R249, I278, Y284, T326, R348, A574, A683, relative to SEQ ID NO: 2. In some embodiments, the variant SHC polypeptide includes an amino acid substitution at 6 or more positions corresponding to positions V45, Q54, Q178, M184,V222, R249, I278, Y284, T326, R348, A574, A683, relative to SEQ ID NO: 2. In some embodiments, the variant SHC polypeptide includes an amino acid substitution at 7 or more positions corresponding to positions V45, Q54, Q178, M184, V222, R249, I278, Y284, T326, R348, A574, A683, relative to SEQ ID NO: 2. In some embodiments, the amino acid substitution is V45L, Q54E, Q178S, M184T, V222Q, V222R, V222K, R249R, I278T, I278S, 1278A, Y284W, T326S, R348R, A574A, A683E, or any combination thereof. In some embodiments, the variant SHC further has at least 60%, 70%, 80%, 90%, 95% but less than 100% sequence identity to SEQ ID NO: 2. In some embodiments, the amino acid substitution is V45L, Q54E, Q178S, M184T, V222Q, V222R, V222K, R249R, I278T, I278S, 1278A, Y284W, T326S, R348R, A574A, A683E, or any combination thereof. In some embodiments, the variant SHC further has at least 70% but less than 100% sequence identity to SEQ ID NO: 2. In some embodiments, the variant SHC further has at least 80% but less than 100% sequence identity to SEQ ID NO: 2. In some embodiments, the variant SHC further has at least 90% but less than 100% sequence identity to SEQ ID NO: 2. In some embodiments, the variant SHC further has at least 95% but less than 100% sequence identity to SEQ ID NO: 2. In some embodiments, the variant SHC further has at least 60%, 70%, 80%, 90%, 95% but less than 100% sequence identity to SEQ ID NO: 1. In some embodiments, the amino acid substitution is V45L, Q54E, Q178S, M184T, V222Q, V222R, V222K, R249R, I278T, I278S, I278A, Y284W, T326S, R348R, A574A, A683E, or any combination thereof. In some embodiments, the variant SHC further has at least 70% but less than 100% sequence identity to SEQ ID NO: 1. In some embodiments, the variant SHC further has at least 80% but less than 100% sequence identity to SEQ ID NO: 1. In some embodiments, the variant SHC further has at least 90% but less than 100% sequence identity to SEQ ID NO: 1. In some embodiments, the variant SHC further has at least 95% but less than 100% sequence identity to SEQ ID NO: 1. In some embodiments, the variant SHC demonstrates increased sclareolide production compared to SEQ ID NO: 2. In some embodiments, the variant SHC demonstrates increased sclareolide production compared to SEQ ID NO: 1.

In some embodiments, SHC variants include amino acid substitutions at positions V45, Q54, I278, and/or T326. Such variants demonstrated increase sclareolide production compared to wild-type enzyme. Particular substitutions include, but are not limited to V45L, Q54E, I278T, and T326S. In some embodiments, the substitutions include V45L, Q54E, I278T, I278S, 1278A, and T326S. In some embodiments, a SHC variant includes amino acid substitutions at one or more (or all) of positions V45, Q54, I278, and/or T326, relative to SEQ ID NO: 2. In some embodiments, a SHC variant includes amino acid substitutions at one or more (or all) of positions V45, Q54, I278, and/or T326, relative to SEQ ID NO: 1. In some embodiments, determining the position relative to SEQ ID NO: 1 includes aligning SEQ ID NO: 1 to SEQ ID NO: 2. In some embodiments, the position in SEQ ID NO: 1 of the substitution corresponds to the position of substitution in SEQ ID NO: 2. For example, if position 19 of SEQ ID NO: 2 is altered, e.g., substituted, the corresponding position in SEQ ID NO: 1 after alignment with SEQ ID NO: 2, position 1, is altered, e.g., substituted. In some embodiments, to determine a position relative to SEQ ID NO: 2, SEQ ID NO: 2 is aligned to the sequence that is or will undergo amino acid alteration.

In particular embodiments, SHC variants include additional amino acid substitutions at positions M184 and V222. In particular embodiments, SHC variants include additional amino acid substitutions at positions Q178, M184, V222, Y284, A683. Such variants demonstrated further increased sclareolide production compared to wild-type enzymes, and those with substitutions at positions V45, Q54, I278, and T326. Particular substitutions include, but are not limited to M184T, V222Q, V222R, and V222K, or combinations, thereof. In some embodiments, the substitutions include, but are not limited to Q178S, M184T, V222Q, V222R, and V222K, Y284W, A683E, or combinations thereof. In some embodiments, the SHC variants include one or more substitutions that are silent mutations. In some embodiments, the SHC variants include 1, 2, 3, 4, 5 or more silent mutations. In some embodiments, the SHC variants include 1, 2, 3, 4, or 5 silent mutations. In some embodiments, the SHC variants include 1, 2, 3, or 4 silent mutations. In some embodiments, the SHC variants include 1, 2, or 3 silent mutations. In some embodiments, the SHC variants include silent mutations at positions 249, 348, 574, or at any one position or a combination thereof. In some embodiments, the silent mutation is R249R, R348R, A574A, or any combination thereof. In some embodiments, the position of the silent mutation is relative to SEQ ID NO: 2. In some embodiments, the silent mutation is at a position in SEQ ID NO: 1 corresponding the position in SEQ ID NO: 2, after alignment of SEQ ID NO: 1 to SEQ ID NO: 2.

In some embodiments, the SHC variants have the following amino acid substitutions:
V45, Q54, I278, T326;
V45, Q54, M184, R249, I278, T326;
V45, Q54, V222, R249, I278, T326;
V45, Q54, R249, I278, T326;
V45, Q54, V222, I278, T326;
V45, Q54, V222, I278, T326;
V45, Q54, V222, I278, T326;
V45, Q54, V222, I278, T326;
V45, Q54, R249, I278, T326;
V45, Q54, R249, I278, T326, A574;
V45, Q54, V222, R249, I278, T326;
V45, Q54, Q178, V222, I278, T326;
V45, Q54, Q178, V222, I278, T326;
V45, Q54, V222, R249, I278, T326, A683;
V45, Q54, V222, R249, I278, Y284, T326;
V45, Q54, V222, I278, T326, R348;
V45, Q54, V222, R249, I278, T326; or
V45, Q54, Q178, V222, I278, T326.

In some embodiments, the SHC variants have the following amino acid substitutions relative to the amino acid sequence of SEQ ID NO: 1:
V45, Q54, I278, T326;
V45, Q54, M184, R249, I278, T326;
V45, Q54, V222, R249, I278, T326;
V45, Q54, R249, I278, T326;
V45, Q54, V222, I278, T326;
V45, Q54, V222, I278, T326;
V45, Q54, V222, I278, T326;
V45, Q54, V222, I278, T326;
V45, Q54, R249, I278, T326;
V45, Q54, R249, I278, T326, A574;
V45, Q54, V222, R249, I278, T326;
V45, Q54, Q178, V222, I278, T326;
V45, Q54, Q178, V222, I278, T326;
V45, Q54, V222, R249, I278, T326, A683;
V45, Q54, V222, R249, I278, Y284, T326;
V45, Q54, V222, I278, T326, R348;
V45, Q54, V222, R249, I278, T326; or
V45, Q54, Q178, V222, I278, T326.

In some embodiments, the variants have the following amino acid substitutions relative to the amino acid sequence of SEQ ID NO: 2:
V45, Q54, I278, T326;
V45, Q54, M184, R249, I278, T326;
V45, Q54, V222, R249, I278, T326;
V45, Q54, R249, I278, T326;
V45, Q54, V222, I278, T326;
V45, Q54, V222, I278, T326;
V45, Q54, V222, I278, T326;
V45, Q54, V222, I278, T326;
V45, Q54, R249, I278, T326;
V45, Q54, R249, I278, T326, A574;
V45, Q54, V222, R249, I278, T326;
V45, Q54, Q178, V222, I278, T326;
V45, Q54, Q178, V222, I278, T326;
V45, Q54, V222, R249, I278, T326, A683;
V45, Q54, V222, R249, I278, Y284, T326;
V45, Q54, V222, I278, T326, R348;
V45, Q54, V222, R249, I278, T326; or
V45, Q54, Q178, V222, I278, T326.

In further particular embodiments, the variants have the following amino acid substitutions relative to the amino acid sequence of SEQ ID NO: 1:
(i) V45L, Q54E, M184T, I278T and T326S,
(ii) V45L, Q54E, V222Q, I278T and T326S,
(iii) V45L, Q54E, I278T and T326S,
(iv) V45L, Q54E, V222R, I278T and T326S,
(v) V45L, Q54E, V222K, I278T and T326S, or
(vi) V45L, Q54E, V222K, I278S and T326S.

In some embodiments, the variants have the following amino acid substitutions:
V45L, Q54E, I278T, T326S;
V45L, Q54E, M184T, R249R, I278T, T326S;
V45L, Q54E, V222Q, R249R, I278T, T326S;
V45L, Q54E, R249R, I278T, T326S;
V45L, Q54E, V222R, I278T, T326S;
V45L, Q54E, V222K, I278T, T326S;
V45L, Q54E, V222K, I278S, T326S;
V45L, Q54E, V222K, I278A, T326S;
V45L, Q54E, R249R, I278A, T326S;
V45L, Q54E, R249R, I278T, T326S, A574A;
V45L, Q54E, V222K, R249R, I278T, T326S;
V45L, Q54E, Q178S, V222K, I278S, T326S;
V45L, Q54E, Q178S, V222R, I278T, T326S;
V45L, Q54E, V222Q, R249R, I278T, T326S, A683E;
V45L, Q54E, V222Q, R249R, I278T, Y284W, T326S;
V45L, Q54E, V222R, I278T, T326S, R348R;
V45L, Q54E, V222K, R249R, I278A, T326S; or
V45L, Q54E, Q178S, V222K, I278T, T326.

In further particular embodiments, the variants have the following amino acid substitutions relative to the amino acid sequence of SEQ ID NO: 1:
V45L, Q54E, I278T, T326S;
V45L, Q54E, M184T, R249R, I278T, T326S;
V45L, Q54E, V222Q, R249R, I278T, T326S;
V45L, Q54E, R249R, I278T, T326S;
V45L, Q54E, V222R, I278T, T326S;
V45L, Q54E, V222K, I278T, T326S;
V45L, Q54E, V222K, I278S, T326S;
V45L, Q54E, V222K, I278A, T326S;
V45L, Q54E, R249R, I278A, T326S;
V45L, Q54E, R249R, I278T, T326S, A574A;
V45L, Q54E, V222K, R249R, I278T, T326S;
V45L, Q54E, Q178S, V222K, I278S, T326S;
V45L, Q54E, Q178S, V222R, I278T, T326S;
V45L, Q54E, V222Q, R249R, I278T, T326S, A683E;
V45L, Q54E, V222Q, R249R, I278T, Y284W, T326S;
V45L, Q54E, V222R, I278T, T326S, R348R;
V45L, Q54E, V222K, R249R, I278A, T326S; or
V45L, Q54E, Q178S, V222K, I278T, T326.

In some embodiments, the position refers to the position in SEQ ID NO: 2 and is the corresponding position in SEQ ID NO: 1 after alignment of SEQ ID NO: 1 to SEQ ID NO: 2.

In some embodiments, the variants have the following amino acid substitutions relative to the amino acid sequence of SEQ ID NO: 2:
(a) V45L, Q54E, I278T, T326S;
(b) V45L, Q54E, M184T, R249R, I278T, T326S;
(c) V45L, Q54E, V222Q, R249R, I278T, T326S;
(d) V45L, Q54E, R249R, I278T, T326S;
(e) V45L, Q54E, V222R, I278T, T326S;
(f) V45L, Q54E, V222K, I278T, T326S;
(g) V45L, Q54E, V222K, I278S, T326S;
(h) V45L, Q54E, V222K, 1278A, T326S;
(i) V45L, Q54E, R249R, I278A, T326S;
(j) V45L, Q54E, R249R, I278T, T326S, A574A;
(k) V45L, Q54E, V222K, R249R, I278T, T326S;
(l) V45L, Q54E, Q178S, V222K, I278S, T326S;
(m) V45L, Q54E, Q178S, V222R, I278T, T326S;
(n) V45L, Q54E, V222Q, R249R, I278T, T326S, A683E;
(o) V45L, Q54E, V222Q, R249R, I278T, Y284W, T326S;
(p) V45L, Q54E, V222R, I278T, T326S, R348R;
(q) V45L, Q54E, V222K, R249R, I278A, T326S; or
(r) V45L, Q54E, Q178S, V222K, I278T, T326.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 12.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 13.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 14.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 15.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 16.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 17.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 18.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 19.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 20.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 21.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 22.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 23.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 24.

In some embodiments, the SHC variant shares at least about 50% sequence identity, at least about 55% sequence identity, at least about 60% sequence identity, at least about 65% sequence identity, at least about 70% sequence identity, at least about 75% sequence identity, at least about 80% sequence identity, at least about 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%, or 100% sequence identity to SEQ ID NO: 25.

In another aspect, nucleic acid molecules that are or contain a nucleic acid sequence encoding an SHC variant are provided. The nucleic acid sequence may encode a particular SHC variant described herein, or an SHC variant having a specified degree of amino acid sequence identity to the particular SHC variant.

In some embodiments, the nucleic acid molecule is or contains a nucleic acid sequence encoding an amino acid sequence having at least 70%, 80%, 90%, 95%, 99%, or 100% sequence identity to the sequence set forth by of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42. In some embodiments, the nucleic acid molecule is or contains a nucleic acid sequence encoding an amino acid sequence having at least 70%, 80%, 90%, 95%, 99%, or 100% sequence identity to the sequence set forth by of SEQ ID NO: 30. In some embodiments, the nucleic acid molecule is or contains a nucleic acid sequence encoding an amino acid sequence having at least 70%, 80%, 90%, 95%, 99%, or 100% sequence identity to the sequence set forth by of SEQ ID NO: 36. In some embodiments, the nucleic acid molecule is or contains a nucleic acid sequence encoding an amino acid sequence having at least 70%, 80%, 90%, 95%, 99%, or 100% sequence identity to the sequence set forth by of SEQ ID NO:37. In some embodiments, the nucleic acid molecule is or contains a nucleic acid sequence encoding an amino acid sequence having at least 70%, 80%, 90%, 95%, 99%, or 100% sequence identity to the sequence set forth by of SEQ ID NO: 42.

In some embodiments, the nucleic acid hybridizes under stringent conditions to a nucleic acid encoding (or complementary to a nucleic acid encoding) an SHC variant having at least 70%, 80%, 90%, 95%, 99%, or 100% sequence identity to the sequence set forth by SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42. In some embodiments, the nucleic acid hybridizes under stringent conditions to a nucleic acid encoding (or complementary to a nucleic acid encoding) an SHC variant having at least 70%, 80%, 90%, 95%, 99%, or 100% sequence identity to the sequence set forth by SEQ ID NO: 30. In some embodiments, the nucleic acid hybridizes under stringent conditions to a nucleic acid encoding (or complementary to a nucleic acid encoding) an SHC variant having at least 70%, 80%, 90%, 95%, 99%, or 100% sequence identity to the sequence set forth by SEQ ID NO: 36. In some embodiments, the nucleic acid hybridizes under stringent conditions to a nucleic acid encoding (or complementary to a nucleic acid encoding) an SHC variant having at least 70%, 80%, 90%, 95%, 99%, or 100% sequence identity to the sequence set forth by SEQ ID NO: 37. In some embodiments, the nucleic acid hybridizes under stringent conditions to a nucleic acid encoding (or complementary to a nucleic acid encoding) an SHC variant having at least 70%, 80%, 90%, 95%, 99%, or 100% sequence identity to the sequence set forth by SEQ ID NO: 42.

Preferably the SHC variants exhibits a better target yield of sclareolide compared to the reference SHC protein. In addition, a SHC variant can exhibit a modified (e.g., increased) target productivity relative to the reference SHC protein. In certain embodiments, a SHC variant exhibits at least a 2-, 3-, 4-, 6-, 8-, 10-, 12-, 14-, 16-, 18-, 20-, 25-, 30-, 35-, 40-, 45-, 50-, 55-, 60-, 65-, 70-, 75-, 80-, 85-, 90-, 95-, or 100-fold increase in enzymatic activity (e.g., conversion of HFA to sclareolide) relative to the reference SHC protein.

To facilitate SHC expression and sclareolide production and isolation, the SHC or SHC variant is expressed in a recombinant host cell. Host cells that may be used for the purposes of this disclosure include, but are not limited to, prokaryotic cells such as bacteria (e.g., E. coli and B. subtilis), which can be transformed with, for example, an expression vector, recombinant bacteriophage DNA, plasmid DNA, bacterial artificial chromosome, or cosmid DNA expression vectors containing the nucleic acid molecules encoding the GmSHC or variant SHCs described herein; simple eukaryotic cells like yeast (for example, Saccharomyces and Pichia), which can be transformed with, for example, recombinant yeast expression vectors containing the polynucleotide molecule of the disclosure; insect cells (e.g., a baculovirus insect cell expression system); human cells (e.g., HeLa, CHO and Jurkat), and plant cells (Arabidopsis and tobacco). Depending on the host cell and the respective vector used to introduce the nucleic acid molecule encoding a GmSHC or variant SHC described herein, the nucleic acid molecule can integrate, for example, into the chromosome or the mitochondrial DNA or can be maintained extrachromosomally, for example, episomally, or can be only transiently harbored by the cell. In embodiments pertaining to a eukaryotic cell, preferably the cell is a fungal, mammalian or plant cell. Suitable eukaryotic cells include, for example, without limitation, mammalian cells, yeast cells (e.g., Saccharomyces, Candida, Kluyveromyces, Schizosaccharomyces, Yarrowia, Pichia and Aspergillus), or insect cells (including Sf9), amphibian cells (including melanophore cells), or worm cells including cells of Caenorhabditis (including Caenorhabditis elegans). Suitable mammalian cells include, for example, without limitation, COS cells (including Cos-1 and Cos-7), CHO cells, HEK293 cells, HEK293T cells, or other transfectable eukaryotic cell lines. In embodiments pertaining to prokaryotes, preferably the cell is E. coli, a Bacillus sp., or Streptomyces sp. In some embodiments, the host cell includes a nucleic acid molecule or expression vector containing a nucleic acid molecule encoding a variant SHC described herein. In some embodiments, the host cell is a yeast, a bacterium, a mammalian cell, or a plant cell. In some embodiments, the host cell is E. coli. In some embodiments, the nucleic acid molecule encoding the variant SHC is codon optimized. While some embodiments include the use of whole intact cells or cell extracts, other embodiments include the use of free, optionally purified or partially purified SHC enzyme or immobilized SHC enzyme for bioconversion of HFA to sclareolide. In this respect, when a soluble wild-type SHC or a SHC variant is used as a biocatalyst, this is considered a two-phase system.

The sclareolide produced by the present two-phase system may be collected, e.g., by steam extraction/distillation or organic solvent extraction using a non-water miscible solvent (to separate the reaction products and unreacted substrate from the biocatalyst which stays in the aqueous phase) followed by subsequent evaporation of the solvent to obtain a crude reaction product as determined by gas chromatographic (GC) analysis.

In some embodiments, sclareolide is produced by contacting a variant SHC described herein or a host cell including an expression vector containing a nucleic acid molecule encoding a variant SHC described herein with HFA. In some embodiments, the HFA includes (3E,7E) HFA. In some embodiments, the contacting occurs in the absence or without addition of metal ions.

The sclareolide may be further selectively crystallized to remove unreacted HFA substrate from the final product. In some embodiments, the isolated crystalline material contains only (−)-sclareolide product. In other embodiments, the isolated crystalline material contains the other isomers, wherein said isomers are present only in olfactory acceptable amounts.

Examples of suitable water miscible and non-water miscible organic solvents suitable for use in the extraction and/or selective crystallization of (−)-sclareolide include, but are not limited to, aliphatic hydrocarbons, preferably those having 5-8 carbon atoms, such as pentane, cyclopentane, hexane, cyclohexane, heptane, octane or cyclooctane; halogenated aliphatic hydrocarbons, preferably those having one or two carbon atoms, such as dichloromethane, chloroform, carbon tetrachloride, dichloroethane or tetrachloroethane; aromatic hydrocarbons, such as benzene, toluene, the xylenes, chlorobenzene or dichlorobenzene; aliphatic acyclic and cyclic ethers or alcohols, preferably those having 4-8 carbon atoms, such as ethanol, isopropanol, diethyl ether, methyl tert-butyl ether, ethyl tert-butyl ether, dipropyl ether, diisopropyl ether, dibutyl ether, tetrahydrofuran; or esters such as ethyl acetate or n-butyl acetate or ketones such as methyl isobutyl ketone or dioxane or mixtures of these. The solvents that are especially preferably used are the above-mentioned heptane, methyl tert-butyl ether (also known as MTBE, tertiary butyl methyl ether and iBME), diisopropyl ether, tetrahydrofuran, ethyl acetate and/or mixtures thereof. Preferably, a water miscible solvent such as ethanol is used for the extraction of (−)-sclarcolide from the solid phase of the reaction mixture. The use of ethanol is advantageous because it is easy to handle, it is non-toxic and it is environmentally friendly.

Desirably, the amount of (−)-sclareolide produced is in the range of about 1 mg/L to about 20,000 mg/L (20 g/L) or higher such as from about 20 g/L to about 200 g/L or from 100 to 200 g/L, preferably about 125 g/L or 150 g/L.

Various applications for (−)-sclarcolide include, but are not limited to, non- enzymatically producing ambrox for use in a fine fragrance or a consumer product such as fabric care, toiletries, beauty care and cleaning products including essentially all products where the currently available ambrox ingredients are used commercially, including but not limited to, AMBROX (Henkel), AMBERLYN (Quest), and norambrenolide ether (Pacific), and other products sold under the trademarks AMBROFIX® (Givaudan), CETALOX® Lacvo (Firmenich), and/or AMBERMOR® (Aromor). In some embodiments, the ambrox produced non-enzymatically from the enzymatically produced sclareolide may be used in a fragrance, e.g., fine fragrance, consumer fragrance, full fragrance, sketch, and/or accord. The selective crystallization of (−)-ambrox may be influenced by the presence of unreacted HFA substrate and also the ratio of (−)-ambrox to the other detectable isomers. In some embodiments, selective crystallization of (−)-ambrox may be influenced by the presence of unreacted HFA and/or byproducts thereof and also the ratio of (−)-ambrox to the other detectable isomers. Even if only 10% conversion of the HFA substrate to enzymatically-produced (−)-sclareolide to non-enzymatically-produced (−)-ambrox is obtained, the selective crystallization of (−)-ambrox is still possible.

Non-enzymatic methods of converting sclareolide to ambrox, e.g., (−)-ambrox include, but are not limited to, methods of reducing sclareolide. In some embodiments, sclareolide may undergo a two-step reaction process for conversion. In some embodiments, the first reaction includes reacting sclareolide with reducing reagents such as, but not limited to, lithium aluminim hydride in a solvent such as, but not limited to, diethyl ether. In some embodiment, the second reaction includes reacting the product of the first step with a reagent such as, but not limited to tosyl chloride in a solvent such as, but not limited to pyridine. Functionalization of C-12 in labdanic diterpenes: synthesis of the natural diterpenic lactones isolated from Cistus ladaniferus L, by Teresa, J. de Pascual; Urones, J. G.; Montana Pedrero, A.; Basabe Barcala, P., Tetrahedron Letters (1985), 26(46), 5717-20, which describes methods of producing ambrox to sclareolide, is incorporated herein by reference in its entirety.

The following non-limiting examples are provided to further illustrate the present invention.

### EXAMPLES

### Example 1: Generation of SHC Variants

Three different approaches were taken to generate SHC variants: rational mutagenesis (site-directed mutagenesis), semi-rational mutagenesis (via site-saturation library), and random mutagenesis (error prone PCR). Variants were expressed in a heterologous system and screened by GC.

Homology Modeling. The three-dimensional structure of GmSHC was built using homology modeling. The templates used were the crystals 1GSZ (Lenhart et al. (2002) Chem. Biol. 9:639-45) and 3SQC (Wendt et al. (1999) J. Mol. Biol. 286:175-87), which share 44% and 43% of sequence identity with 95% of GmSHC sequence.

Molecular Docking. The ground state representations of HFA were then docked to the active center of the GmSHC structure. This was achieved by defining a three-dimensional grid box centered in the protonated oxygen atom of the first proton donor. This grid box identifies the active center pocket area where the substrates conformations will be sampled during the molecular docking run. Then molecular docking was performed using the Lamarckian genetic algorithm (LGA; Morris et al. (1998) J. Comput. Chem. 19:1639-62; Morris et al. (2009) J. Comput. Chem. 30:2785-91). A total of 1000 LGA runs were carried out per system. The population was 300, the GA elitism=1, the maximum number of generations was 27,000 and the maximum number of energy evaluations was 2,500,000. Accordingly, for each LGA run the first generation started with a population of 300 random substrates conformations. The best substrate conformation in the current population automatically survives into the next generation (GA elitism=1). As such, the next generation population starts with the fittest substrate conformation from the previous generation plus another 299 conformations. The LGA run stops when the number of maximum generations or energy evaluations are reached. For each LGA run, one substrate conformation was obtained. Substrate conformations were then sorted according to energy and root mean square deviation. The top ranked structure corresponded to the lowest binding energy structure of the most populated cluster with the lowest mean binding energy.

SHC Structural Analysis and Catalytic Mechanism. SHCs are integral monotopic membrane proteins which adopt a dimeric 3D arrangement. Each monomer is characterized by eight QW motifs (Sato et al. (1998) Biosci. Biotechnol. Biochem. 62:407-11) that tightly connect numerous a-helices building up two highly stable α/α-barrels domains (Wendt et al. (1999) J. Mol. Biol. 286:175-87). The active center cavity is buried within the two a/a-barrels domains and its access is possible through an inner hydrophobic channel. For AaSHC, the channel and the active center cavity are separated by a narrow constriction constituted by residues F166, V174, F434, and C435, which is responsible for substrate recognition (Lenhart et al. (2002) Chem. Biol. 9:639-45). For GmSHC, those residues correspond to F176, M184, F457 and C458. Unless indicated otherwise, the position of the amino acid residues provided with respect to GmSHC are with reference to SEQ ID NO: 2. At the top of the activity center cavity, the residues that constitute the conserved DXDD motif (Wendt et al. (1999) J. Mol. Biol. 286:175-87) are observed. One of those residues is D396, the first proton donor, which initiates the cyclization by donating a proton to the double bond 2 and 3 (Scheme 2). In GmSHC, the oxygen atom of D396 is 4.6 Ä from the carbon 3 of the double bond 2,3.

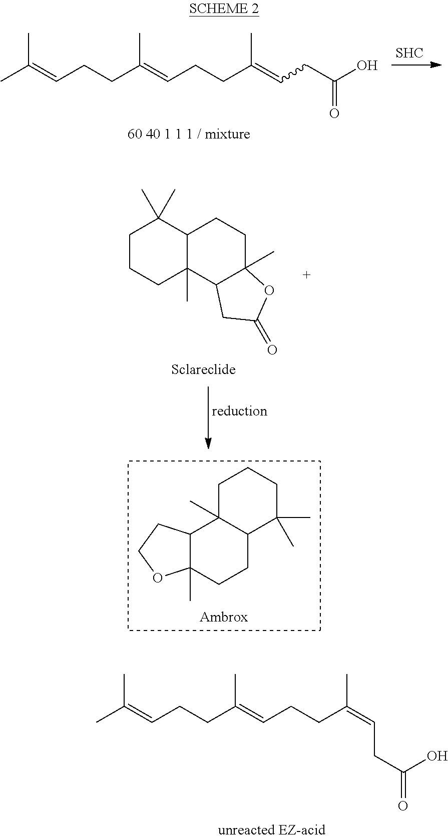

The DXDD motif is followed by tryptophan and phenylalanine residues that are responsible for stabilizing the cationic intermediates by strong cation-π interactions (Dougherty (1996) Science 271:163-168). On the bottom of the cavity is a glutamate residue, the last proton acceptor, which may receive a proton from the hydroxyl group and lead to the closing of the third ring and formation of the product, sclareolide. Structural analysis of GmSHC indicates that this enzyme possesses two possible last proton acceptors. However, according to the docking results, E386 of GmSHC is the most likely the last proton acceptor. The distance between the sclareolide hydroxyl oxygen and E386 is just 3.5 Ä. Clearly this disposition of the last proton acceptor plays an important role in the catalytic efficacy of this enzyme.

Using the molecular model, GmSHC residues that establish the cation-pi interactions responsible for the stabilization of the cationic intermediate and the other main catalytic residues were determined. Notably, most of the important catalytic residues are conserved with other RpSHC enzyme (WO 2010/139719). The main differences include: (a) the GmSHC active center is residue 45; (b) GmSHC residue 184, together with residues 176, 457 and 458, is responsible for the narrow constriction between the hydrophobic channel and the active center cavity, which is associated with substrate selectivity; and (c) the pattern of QW motifs is somewhat different.

Structural Hotspots. Based on the molecular modeling and molecular docking results, the following active center structural hotspots, which when mutated can improve the chemical step of the enzyme catalysis, were identified: residues V45, E46, Q54, F176, M184, F457, C458, W179, I278, Q279, T326, F385, E386, D397, F443, F460, F624, F654 and L656. Position V222 was also identified.

Specificity Determining Positions and Conserved Residues. The specificity determining positions indicate which residues coordinately evolved within a subgroup of proteins of a family that shares a given catalytic specificity. Thus, it allows following the evolutionary process associated with acquiring a diversity of biological functions within the same family of proteins. Specificity determining positions were calculated from a multiple sequence alignment containing 1000 homologous sequences using the algorithms of Xdet.

Evolution of GmSHC. To improve the catalytic conversion of HFA to sclareolide, GmSHC was modified to (1) improve the Michaelis-Menten complex; (2) introduce mutations that can increase the cation-π stabilization of the carbocation intermediate, based on the structural and coevolution hotspots; (3) open the catalytic cavity by mutating the residues that are only essential for the catalysis of the 5-ring native substrate, squalene; (4) mutate the residues that assist the last proton acceptor in order to facilitate product formation; (5) alter the active center; (6) mutate residues responsible for the narrow constriction between the hydrophobic channel and the active center cavity; (7) and increase the QW motifs.

GmSHC variants designed to improve the Michaelis-Menten complex, and increase the cation-π stabilization of the carbocation intermediate, open the catalytic cavity by mutating the residues that are only essential for the catalysis of the 5-ring native substrate, squalene, mutate the residues that assist the last proton acceptor in order to facilitate product formation and alter the active center were tested in silico using molecular docking. The results of this analysis are presented in Table 2.

TABLE 2
GmSHC modified to improve the Michaelis-Menten complex

| | Inhibition Constant | |
|---|---|---|
| SHC | Ki (μM) | Comments |
| WT | 283.78 | — |
| I278F | 114.79 | The substrate is more distant from the first |
| | | proton donor than in WT. |
| V45I | 412.35 | The substrate is more distant from the first |
| | | proton donor than in WT. |
| V45L | 127.22 | Similar to WT. L45 interacts directly with the |
| | | substrate, which increases the substrate binding |
| | | affinity. |
| L656W | 187.80 | Substrate is closer to the |
| | | first proton donor than |
| | | in WT; |
| | | Substrate is closer to the last proton acceptor |
| | | than in WT. |
| L656F | 197.06 | Substrate is closer to the first proton donor than |
| | | in WT; |
| | | Substrate is closer to the last proton acceptor |
| | | than in WT. |
| W179A | 320.26 | The substrate is more distant from the first |
| | | proton donor. |
| I278A | 158.80 | Similar to WT |
| F654Y | 155.01 | Designed to increase cation-π interactions. |
| | | Substrate pose similar to WT. |
| I278T | 92.96 | Similar to WT. |
| | | The substrate carboxylic moiety interacts with |
| | | T278. T278 assists the last proton acceptor. |
| I278S | 241.75 | The substrate carboxylic moiety interacts with |
| | | S278. S278 assists the last proton acceptor. The |
| | | substrate is closer to the last proton acceptor |
| | | than in WT. |
| T326S | 267.72 | The substrate is closer to the last proton |
| | | acceptor than in WT. The substrate carboxylic |
| | | moiety interacts with S326. S326 assists the |
| | | last proton acceptor. |
| L656E | 188.69 | L656E is the last proton acceptor. |
| Q54E | 221.01 | The substrate is closer to the first proton donor. |
| F460A | 214.98 | Opens the lower part of the active center cavity. |
| | | The substrate is more distant from the last |
| | | proton acceptor than in WT. |
| F443Y | 239.72 | Designed to increase cation-π interactions. The |
| | | substrate is more distant from the last proton |
| | | acceptor than in WT. |
| V45L + T326S | 179.31 | Similar to WT. |
| | | S326 and L45 interact directly with the |
| | | substrate, which increases the substrate binding |
| | | affinity. S326 assists the last proton acceptor. |

Additional mutations addressing each of the modifications indicated above are listed Table 3.

TABLE 3
Additional GmSHC modified to improve the Michaelis-Menten complex

| Functional Change | GmSHC Mutation |
|---|---|
| (1) improve the Michaelis-Menten complex; and | V45I |
| (5) alter the active center | V45L |
| | V45Q |
| | V45A |
| | E46Q |
| | E46A |
| | E46H |
| | Q54E |
| | Q54H |
| | G623A |
| | G623V |
| | F385Y |
| | E386D |
| | E386Q |
| | E386H |
| | E386N |
| | F443I |
| | F443L |
| | F443A |
| | F443V |
| | F443H |
| | F443Y |
| | F654A |
| | F654L |
| | F624A |
| | L656I |
| | L656E |
| | L656N |
| | V45Q + L656E |
| | V45L + T326S |
| | T326D + E386T |
| | F385Y + F654Y |
| (2) introduce mutations that can increase the cation-π | F385Y |
| stabilization of the carbocation intermediate, based on the | F443Y |
| structural and coevolution hotspots | F624W |
| | F624Y |
| | F654W |
| | F654Y |
| | L656F |
| | L656Y |
| | L656W |
| | F385Y + F654Y |
| | F385Y + F654Y + F443Y |
| (1) improve the Michaelis-Menten complex; and | W179A |
| (3) open the catalytic cavity by mutating the residues that are | W179V |
| only essential for the catalysis of the 5-ring native substrate, | |
| squalene | |
| (1) improve the Michaelis-Menten complex; and | T326E |
| (4) mutate the residues that assist the last proton acceptor in | T326D |
| order to facilitate product formation | T326S |
| | T326N |
| | T326C |
| | T326N + 1278N |
| (1) improve the Michaelis-Menten complex; | I278A |
| (3) open the catalytic cavity by mutating the residues that are | I278V |
| only essential for the catalysis of the 5-ring native substrate, | I278F |
| squalene; and | I278Y |
| (4) mutate the residues that assist the last proton acceptor in | I278T |
| order to facilitate product formation | I278S |
| | I278N |
| (5) alter the active center | Q178H |
| | Q178S |
| | Q178E |
| | Q178D |
| | D397C |
| | D397C + D394V + V471C |
| (6) mutate residues responsible for the narrow constriction | M184L |
| between the hydrophobic channel and the active center cavity | M184V |
| | M184I |
| | M184C |
| | M184T |
| | M184A |
| (7) and increase the QW motifs | R194Q |
| | M305W |
| | S321W |
| | S321F |
| | V222Q |
| | P412Q |
| | M345W |
| Other | W87L |
| | W87V* |
| | V222K |
| | V222R |
| | L335F |
| | S321F |
| | F460A |
| | F460H |
| | F460L |
| | Y658F |
| | W556A |
| | W556V* |
| | Q279V + Q54V |
| | D397C + D394V |
| | F385H + E386A |
| | F385E + E386A |
| | F385Y + F654Y + F443Y |

*From the literature.

SHC Variant Enzyme Expression. Wild-type and the GmSHC variants of Table 5 were individually cloned into pET28a(+). These DNA constructs were transformed into Lemo21 (DE3) E. coli and plated onto agar plates containing Kanamycin. These were incubated overnight at 37° C. A single bacterial colony was picked and used to inoculate 10 mL LB+Kanamycin in a 50 mL falcon tube. This primary culture was incubated overnight at 37° C. with agitation. These cultures (10 mL) were used to inoculate 1L LB+Kanamycin in a shake flask, which was subsequently incubated at 37° C. at 180 rpm for about 4 hours. Protein expression was then induced with the addition of 0.4 mM IPTG and 0.2 mM L-rhamnose. The incubator temperature was lowered to 25° C. and the cultures further incubated at 180 rpm overnight. The next day, the cultures were centrifuged at 4000 rpm for 10 minutes and the supernatant discarded. Cell pellets were resuspended in 0.1 M Potassium phosphate buffer pH 7.4 and exposed to 1 round of freeze/thawing before lyophilization and then use in the reaction assay.

Cell pellet (1 μL) was spotted onto a nitrocellulose membrane and allowed to air dry for 30 minutes. The membrane was placed into 5% milk powder for 1 hour at room temperature with gentle agitation. The membrane was then rinsed with phosphate-buffered saline (PBS), 3×5 minutes. Anti-histidine antibody solution (1 in 10,000 dilution) was added and incubated at room temperature for 1 hour with shaking. The blot was subsequently washed in PBS, 3×5 minutes. Developing solution (6 mg diaminobenzidine (DAB) and 5 μL 30% H₂O₂ in 10 mL PBS) was added to the blot. Once developed, the developing solution was immediately removed, and the blot rinsed with water.

The results of this analysis indicated that all constructs were expressed in E. coli at 25° C. following the addition of 1 mM IPTG. Following expression and processing of the enzymes, a dot blot was performed to assess if the introduction of specific mutations had altered the protein expression. Notably, the majority of the GmSHC variants showed similar levels of expression to the wild-type GmSHC construct.

SHC Variant Screening Reactions. Sodium citrate buffer 0.2 M, pH 5 was prepared and 500 μL of this buffer was added to freeze-dried whole cells of each variant (from 1 L shake flask fermentation of E. coli transformed with desired variant plasmid). The results were obtained with an enzyme loading of 50% (w/w). Subsequently, HFA (20 mg/mL) was added to the buffer/enzyme mix. The reactions were incubated at 25° C. with agitation for 72 hours. To stop the reaction and extract the products, a 2X volume of 3:2, heptane: isopropanol was added to each reaction. These were then incubated at 37° C. for 30 minutes with agitation to mix thoroughly. The reactions were centrifuged for 10 minutes at 4000 rpm to pellet any cellular material. The upper organic layer was then removed and placed in a clean gas chromatography (GC) vial.

GC Analysis Method. A GC analytical method was used to detect each of the starting materials and products used in the screening reactions. Due to the volume of samples generated, a fast method was developed with a run time of only 4.5 minutes. The GC analysis conditions are presented in Table 4.

TABLE 4
Starting materials and products used in the screening reactions

| Component | Condition | | |
|---|---|---|---|
| GC system | Perkin Elmer Autosystem XL | | |
| Column | Agilent HP-5 (30 m, 0.25 mm × 0.25 μm) | | |
| Carrier gas | Helium | | |
| Carrier pressure | 30 psi | | |
| Oven program | Rate/° C. min⁻¹ | Temperature/° C. | Hold/min |
| | 0 | 200 | 1.5 |
| | 45 | 225 | 0.5 |

| | |
|---|---|
| Injection temperature | 270° C. |
| Detector | FID |
| Detector temperature | 270° C. |
| Injection volume | 1 μL |
| Syringe volume | 10 μL |
| Data acquisition time | 4.5 minutes |

Analysis of the SHC variant reaction samples following the addition of 10 mg/mL HFA with 100% enzyme loading indicated that a number of SHC variants exhibited improved activity (based on % peak area) compared to the wild-type enzyme (Table 5). The wild-type enzyme did not show any conversion of HFA to sclareolide and therefore all fold changes were calculated based on the first mutant enzyme which showed formation of sclareolide, i.e., the V45L+Q54E+I278T+T326S variant. No metal ions were included in the reaction.

TABLE 5
SHC variants with improved sclareolide production

| | Fold |
|---|---|
| Mutations | improvement |
| V45L + Q54E + I278T + T326S | -0- |
| V45L + Q54E + M184T + R249R + I278T + T326S | 3.3 |
| V45L + Q54E + V222Q + R249R + I278T + T326S | 3.9 |
| V45L + Q54E + R249R + I278T + T326S | 4.2 |
| V45L + Q54E + V222R + I278T + T326S | 4.2 |
| V45L + Q54E + V222K + I278T + T326S | 5.4 |
| V45L + Q54E + V222K + I278S + T326S | 7.4 |

Further analysis was conducted to identify one or more optimal SHC enzymes. In particular, substrate loading was increased to 20 mg/mL and incubation with a reduced enzyme loading (50%). As shown in Table 6, there were multiple variant enzymes which displayed higher activity than the wild-type and initial mutant SHC enzyme.

TABLE 6
SHC variants (50% enzyme loading) with
improved sclareolide production.

| | Fold |
|---|---|
| Mutations | improvements |
| V45L + Q54E + I278T + T326S | 0 |
| V45L + Q54E + R249R + I278T + T326S | 7 |
| V45L + Q54E + V222K + I278T + T326S | 12 |
| V45L + Q54E + V222K + I278A + T326S | 20.4 |
| V45L + Q54E + R249R + I278A + T326S | 25.6 |
| V45L + Q54E + V222R + I278T + T326S | 27 |
| V45L + Q54E + R249R + I278T + T326S + A574A | 27 |
| V45L + Q54E + V222K + I278S + T326S | 28.4 |
| V45L + Q54E + V222Q + R249R + I278T + T326S | 30.3 |
| V45L + Q54E + V222K + R249R + I278T + T326S | 33.2 |
| V45L + Q54E + Q178S + V222K + I278S + T326S | 37.3 |
| V45L + Q54E + Q178S + V222R + 1278T + T326S | 43.8 |
| V45L + Q54E + V222Q + R249R + I278T + T326S + | 44.6 |
| A683E | |
| V45L + Q54E + V222Q + Y284W + R249R + 1278T + | 46.2 |
| T326S | |
| V45L + Q54E + V222R + I278T + T326S + R348R | 60 |

### Example 2: Summary of GmSHC Variants

SHCs are integral monotopic membrane proteins that adopt a dimeric three-dimensional arrangement. Each monomer is characterized by QW motifs that tightly connect numerous a-helices building up two highly stable α/α-barrels domains (Wendt et al. (1999) J. Mol. Biol. 286:175-87). The active center cavity is buried within the two a/a-barrels domains and its access is possible through an inner hydrophobic channel, which it is suggested to be the membrane-immersed region of the enzyme (Lenhart et al. (2002) Chem. Biol. 9:639-45). The channel and the active center cavity are separated by a narrow constriction which is responsible for substrate recognition (Lenhart et al. (2002) Chem. Biol. 9:639-45). For GmSHC, those residues correspond to Phe176, Met184, Phe457 and Cys458. The residues that constitute the conserved DXDD motif (Wendt et al. (1999) J. Mol. Biol. 286:175-87), are found at the top of the activity center cavity. One of those residues is Asp396, the first proton donor, which initiates cyclization of homofarnesol by donating a proton to the double bond C2═C3. The DXDD motif is followed by tryptophan, tyrosine and phenylalanine residues that are responsible for stabilizing the cationic intermediates by strong cation-π interactions (Dougherty (1996) Science 271:163-8). On the bottom of the cavity is a negatively charged residue, the last proton acceptor, which receives a proton from the hydroxyl group thereby resulting in closure of the third ring and formation of ambrox.

To improve the conversion of HFA to sclareolide, GmSHC was mutated at one or more of the residues at position 45, 54, 178, 184, 222, 278, 284, 326, 348, 574 and 683 of SEQ ID NO: 2. Silent mutations at positions 249, 348, and 574 identified in an error prone library, were also introduced in the GmSHC of SEQ ID NO: 2.

Positions 45 and 326. According to the GmSHC homology model and molecular docking calculations, residues V45 and T326 are placed near the substrate hydroxyl group. GmSHC position 45 is mutated to glutamine, leucine or isoleucine and position 326 to serine in order to increase the intermolecular interactions with the substrate. In some cases, position 45 was mutated to leucine. In some cases, position 326 was mutated to serine.

Positions 54 A structural alignment between AaSHC (Reinhert, et al. (2004) Chem. Biol. 11:121-6) and the GmSHC homology model indicates that Q54 of GmSHC is superimposed with residue E45 of AaSHC, which is the last proton acceptor of this enzyme (Dang & Prestwich (2000) Chem. Biol. 7:643-9). Therefore, residue 54 of GmSHC was mutated to glutamate to incorporate a last proton acceptor at this position, without having a negative impact on the charge network associated with the conserved DXDD motif.

Position 178. A structural alignment between the GmSHC homology model and the homologous human lanosterol synthase (Thoma et al. (2004) Nature 432:118-22) indicates that Q178 of GmSHC is superimposed with residue H232 of the human lanosterol synthase, which is the last proton acceptor of this enzyme. Therefore, residue 178 of GmSHC was mutated to glutamate to incorporate a last proton acceptor at this position. Q178S was selected by site-saturation mutagenesis for improving activity.

Position 184. Residue M184 is placed in narrow constriction, which is responsible for substrate recognition (Lenhart et al. (2002) Chem. Biol. 9:639-45). Thus, to alter the substrate recognition, M184 of GmSHC was mutated to non-polar amino acids, i.e., Leu, Ile, Val and Ala. By mutating this position, any methionine oxidation phenomenon is also prevented, which could negatively affect substrate recognition. It was observed by site saturation mutagenesis that mutant M184T resulted in an increase in activity.

Position 278. According to the GmSHC homology model and the molecular docking calculations, residue I278 is placed right below the substrate hydroxyl group. When residue I278 is mutated to valine, the molecular docking calculations indicate that the substrate arrangement within the active center improves by placing the substrate C2═C3 double bond closer to D396, the first proton donor. Mutant I278A opens the catalytic cavity near the last proton acceptor. Mutant I278T interacts with the substrate and assists the last proton acceptor in the final product formation. When residue I278 is mutated to serine, the molecular docking calculations indicate that it does not just interact with the last proton acceptor but also promotes the substrate arrangement within the active center placing it closer to the last proton donor.

Position 222. Each SHC monomer is characterized by QW motifs that tightly connect numerous α-helices building up two highly stable a/a-barrels domains (Wendt et al. (1999) J. Mol. Biol. 286:175-87). Variant V222Q was designed to establish a new QW motif with W229, which further increases the structural stability of the enzyme. In a sequence alignment of 1,000 homologous enzymes of Gluconobacter morbifer SHC (GmSHC), the consensus residue for position 222 is arginine. Consensus residues, like V222R, are typically associated with a higher structural stability (Steipe et al. (1994) J. Mol. Biol. 240(3):188-92). Therefore, mutation of V222 to Q or R was expected to improve activity. It was subsequently found that in fact K at this position was most beneficial.

Position 249. R249R was identified in an error prone library. This residue interacts with the cell membrane. This residue is placed in the enzyme: membrane interface.

Position 284. Like F176, M184, F457 and C458, Y284 is also placed in a narrow constriction that separates the channel from the active center pocket, which is responsible for substrate recognition. It was observed by site saturation mutagenesis that variant Y284W increases activity, most likely by being s able to increase the specificity of the enzyme for HFA.

Position 348. R348R was identified in an error prone library and it is placed in a flexible loop of the enzyme.

Position 574. A574A was identified in an error prone library. This silent mutation is placed in a flexible loop.

Position 683. A683E is placed in the C-terminal domain of the enzyme and introduces a negative charged residue in this region, which have a positive effect on activity.

1. A variant Squalene Hopene Cyclase (SHC) polypeptide having at least 60% sequence identity to SEQ ID NO: 2 and comprising an amino acid substitution at one or more positions corresponding to positions V45, Q54, Q178, M184, V222, R249, I278, Y284, T326, R348, A574, or A683, relative to SEQ ID NO: 2, wherein the variant demonstrates increased sclareolide production compared to SEQ ID NO: 2.

2. The variant SHC of claim 1, wherein the amino acid substitution at the one or more positions is V45L, Q54E, Q178S, M184T, V222Q, V222R, V222K, R249R, I278T, I278S, I278A, Y284W, T326S, R348R, A574A, A683E, or any combination thereof.

3. The variant SHC of claim 1, wherein the variant SHC polypeptide comprises amino acid substitutions:
(a) V45L, Q54E, I278T, T326S;
(b) V45L, Q54E, M184T, R249R, I278T, T326S;
(c) V45L, Q54E, V222Q, R249R, I278T, T326S;
(d) V45L, Q54E, R249R, I278T, T326S;
(e) V45L, Q54E, V222R, I278T, T326S;
(f) V45L, Q54E, V222K, I278T, T326S;
(g) V45L, Q54E, V222K, I278S, T326S;
(h) V45L, Q54E, V222K, I278A, T326S;
(i) V45L, Q54E, R249R, I278A, T326S;
(j) V45L, Q54E, R249R, I278T, T326S, A574A;
(k) V45L, Q54E, V222K, R249R, I278T, T326S;
(l) V45L, Q54E, Q178S, V222K, I278S, T326S;
(m) V45L, Q54E, Q178S, V222R, I278T, T326S;
(n) V45L, Q54E, V222Q, R249R, I278T, T326S, A683E;
(o) V45L, Q54E, V222Q, R249R, I278T, Y284W, T326S;
(p) V45L, Q54E, V222R, I278T, T326S, R348R;
(q) V45L, Q54E, V222K, R249R, I278A, T326S; or
(r) V45L, Q54E, Q178S, V222K, I278T, T326S.

4. The variant SHC of claim 1, wherein the variant SHC polypeptide has at least 70%, 80%, 90%, 95%, 99%, or 100% sequence identity to the sequence set forth by SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

5. A nucleic acid molecule comprising a nucleic acid sequence encoding a variant SHC polypeptide according to claim 1.

6. The nucleic acid molecule of claim 5, comprising a nucleic acid sequence that:
i) encodes an amino acid sequence having at least 70%, 80%, 90%, 95%, 99%, or 100% sequence identity to the sequence set forth by SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25;
ii) has at least 70%, 80%, 90%, 95%, 99%, or 100% sequence identity to the sequence set forth by SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42; or
iii) hybridizes under stringent conditions to a nucleic acid sequence having a sequence complementary to the sequence set forth by SEQ ID NO: 27, SEQ ID NO: 28, SEQ ID NO: 29,SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42.

7. The nucleic acid molecule of claim 5, comprising a heterologous regulatory sequence, optionally a promoter sequence.

8. An expression vector comprising a nucleic acid molecule encoding a variant SHC polypeptide of claim 1 or a nucleic acid molecule of claim 5.

9. A recombinant host cell comprising a nucleic acid molecule of claim 5 or an expression vector of claim 8.

10. A method for producing sclareolide comprising contacting homofarnesoic acid with a variant SHC polypeptide of claim 1 recombinant host cell of claim 9.

11. The method of claim 10, comprising collecting the sclareolide.

12. The method of claim 10, wherein the homofarnesoic acid comprises (3E,7E) homofarnesoic acid.

13. The method of claim 10, comprising non-enzymatically converting the sclareolide to ambrox.


SEQUENCE LISTING (42 sequences)

SEQ ID NO: 1 - protein, 667 aa, Gluconobacter morbifer
MLPEAVSSACDWLIDQQKPDGHWVGPVESNACMEAQWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGIQPPWIYGLMALSVEGYPLHHPVLAKAMDALNDPGWRRDKGDASWIQATNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 2 - protein, 685 aa, Gluconobacter morbifer
MSPADISTKSSSFQRLDNMLPEAVSSACDWLIDQQKPDGHWVGPVESNACMEAQWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGIQPPWIYGLMALSVEGYPLHHPVLAKAMDALNDPGWRRDKGDASWIQATNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 3 - protein, 21 aa, synthetic construct
GLNXAAXAAXAAGLYXAATRP

SEQ ID NO: 4 - protein, 18 aa, synthetic construct
ASPXAAASPASPTHRALA

SEQ ID NO: 5 - protein, 2175 aa, Zymomonas mobilis
METGLYILEASPARGMETASNSERLEUSERARGLEULEUMETLYSLYSILEPHEGLYALAGLULYSTHRSERTYRLYSPROALASERASPTHRILEILEGLYTHRASPTHRLEULYSARGPROASNARGARGPROGLUPROTHRALALYSVALASPLYSTHRILEPHELYSTHRMETGLYASNSERLEUASNASNTHRLEUVALSERALACYSASPTRPLEUILEGLYGLNGLNLYSPROASPGLYHISTRPVALGLYALAVALGLUSERASNALASERMETGLUALAGLUTRPCYSLEUALALEUTRPPHELEUGLYLEUGLUASPHISPROLEUARGPROARGLEUGLYASNALALEULEUGLUMETGLNARGGLUASPGLYSERTRPGLYVALTYRPHEGLYALAGLYASNGLYASPILEASNALATHRVALGLUALATYRALAALALEUARGSERLEUGLYTYRSERALAASPASNPROVALLEULYSLYSALAALAALATRPILEALAGLULYSGLYGLYLEULYSASNILEARGVALPHETHRARGTYRTRPLEUALALEUILEGLYGLUTRPPROTRPGLULYSTHRPROASNLEUPROPROGLUILEILETRPPHEPROASPASNPHEVALPHESERILETYRASNPHEALAGLNTRPALAARGALATHRMETVALPROILEALAILELEUSERALAARGARGPROSERARGPROLEUARGPROGLNASPARGLEUASPGLULEUPHEPROGLUGLYARGALAARGPHEASPTYRGLULEUPROLYSLYSGLUGLYILEASPLEUTRPSERGLNPHEPHEARGTHRTHRASPARGGLYLEUHISTRPVALGLNSERASNLEULEULYSARGASNSERLEUARGGLUALAALAILEARGHISVALLEUGLUTRPILEILEARGHISGLNASPALAASPGLYGLYTRPGLYGLYILEGLNPROPROTRPVALTYRGLYLEUMETALALEUHISGLYGLUGLYTYRGLNLEUTYRHISPROVALMETALALYSALALEUSERALALEUASPASPPROGLYTRPARGHISASPARGGLYGLUSERSERTRPILEGLNALATHRASNSERPROVALTRPASPTHRMETLEUALALEUMETALALEULYSASPALALYSALAGLUASPARGPHETHRPROGLUMETASPLYSALAALAASPTRPLEULEUALAARGGLNVALLYSVALLYSGLYASPTRPSERILELYSLEUPROASPVALGLUPROGLYGLYTRPALAPHEGLUTYRALAASNASPARGTYRPROASPTHRASPASPTHRALAVALALALEUILEALALEUSERSERTYRARGASPLYSGLUGLUTRPGLNLYSLYSGLYVALGLUASPALAILETHRARGGLYVALASNTRPLEUILEALAMETGLNSERGLUCYSGLYGLYTRPGLYALAPHEASPLYSASPASNASNARGSERILELEUSERLYSILEPROPHECYSASPPHEGLYGLUSERILEASPPROPROSERVALASPVALTHRALAHISVALLEUGLUALAPHEGLYTHRLEUGLYLEUSERARGASPMETPROVALILEGLNLYSALAILEASPTYRVALARGSERGLUGLNGLUALAGLUGLYALATRPPHEGLYARGTRPGLYVALASNTYRILETYRGLYTHRGLYALAVALLEUPROALALEUALAALAILEGLYGLUASPMETTHRGLNPROTYRILETHRLYSALACYSASPTRPLEUVALALAHISGLNGLNGLUASPGLYGLYTRPGLYGLUSERCYSSERSERTYRMETGLUILEASPSERILEGLYLYSGLYPROTHRTHRPROSERGLNTHRALATRPALALEUMETGLYLEUILEALAALAASNARGPROGLUASPTYRGLUALAILEALALYSGLYCYSHISTYRLEUILEASPARGGLNGLUGLNASPGLYSERTRPLYSGLUGLUGLUPHETHRGLYTHRGLYPHEPROGLYTYRGLYVALGLYGLNTHRILELYSLEUASPASPPROALALEUSERLYSARGLEULEUGLNGLYALAGLULEUSERARGALAPHEMETLEUARGTYRASPPHETYRARGGLNPHEPHEPROILEMETALALEUSERARGALAGLUARGLEUILEASPLEUASNASN

SEQ ID NO: 6 - protein, 2052 aa, Bradyrhizobium sp.
METTHRVALTHRSERSERALASERALAARGALATHRARGASPPROGLYASNTYRGLNTHRALALEUGLNSERTHRVALARGALAALAALAASPTRPLEUILEALAASNGLNLYSPROASPGLYHISTRPVALGLYARGALAGLUSERASNALACYSMETGLUALAGLNTRPCYSLEUALALEUTRPPHEMETGLYLEUGLUASPHISPROLEUARGLYSARGLEUGLYGLNSERLEULEUASPSERGLNARGPROASPGLYALATRPGLNVALTYRPHEGLYALAPROASNGLYASPILEASNALATHRVALGLUALATYRALAALALEUARGSERLEUGLYPHEARGASPASPGLUPROALAVALARGARGALAARGGLUTRPILEGLUALALYSGLYGLYLEUARGASNILEARGVALPHETHRARGTYRTRPLEUALALEUILEGLYGLUTRPPROTRPGLULYSTHRPROASNILEPROPROGLUVALILETRPPHEPROLEUTRPPHEPROPHESERILETYRASNPHEALAGLNTRPALAARGALATHRLEUMETPROILEALAVALLEUSERALAARGARGPROSERARGPROLEUPROPROGLUASNARGLEUASPALALEUPHEPROHISGLYARGLYSALAPHEASPTYRGLULEUPROVALLYSALAGLYALAGLYGLYTRPASPARGPHEPHEARGGLYALAASPLYSVALLEUHISLYSLEUGLNASNLEUGLYASNARGLEUASNLEUGLYLEUPHEARGPROALAALATHRSERARGVALLEUGLUTRPMETILEARGHISGLNASPPHEASPGLYALATRPGLYGLYILEGLNPROPROTRPILETYRGLYLEUMETALALEUTYRALAGLUGLYTYRPROLEUASNHISPROVALLEUALALYSGLYLEUASPALALEUASNASPPROGLYTRPARGVALASPVALGLYASPALATHRTYRILEGLNALATHRASNSERPROVALTRPASPTHRILELEUTHRLEULEUALAPHEASPASPALAGLYVALLEUGLYASPTYRPROGLUALAVALASPLYSALAVALASPTRPVALLEUGLNARGGLNVALARGVALPROGLYASPTRPSERMETLYSLEUPROHISVALLYSPROGLYGLYTRPALAPHEGLUTYRALAASNASNTYRTYRPROASPTHRASPASPTHRALAVALALALEUILEALALEUALAPROLEUARGHISASPPROLYSTRPLYSALALYSGLYILEASPGLUALAILEGLNLEUGLYVALASPTRPLEUILEGLYMETGLNSERGLNGLYGLYGLYTRPGLYALAPHEASPLYSASPASNASNGLNLYSILELEUTHRLYSILEPROPHECYSASPTYRGLYGLUALALEUASPPROPROSERVALASPVALTHRALAHISILEILEGLUALAPHEGLYLYSLEUGLYILESERARGASNHISPROSERMETVALGLNALALEUASPTYRILEARGARGGLUGLNGLUPROSERGLYPROTRPPHEGLYARGTRPGLYVALASNTYRVALTYRGLYTHRGLYALAVALLEUPROALALEUALAALAILEGLYGLUASPMETTHRGLNPROTYRILEGLYARGALACYSASPTRPLEUVALALAHISGLNGLNALAASPGLYGLYTRPGLYGLUSERCYSALASERTYRMETASPVALSERALAVALGLYARGGLYTHRTHRTHRALASERGLNTHRALATRPALALEUMETALALEULEUALAALAASNARGPROGLNASPLYSASPALAILEGLUARGGLYCYSMETTRPLEUVALGLUARGGLNSERALAGLYTHRTRPASPGLUPROGLUPHETHRGLYTHRGLYPHEPROGLYTYRGLYVALGLYGLNTHRILELYSLEUASNASPPROALALEUSERGLNARGLEUMETGLNGLYPROGLULEUSERARGALAPHEMETLEUARGTYRGLYMETTYRARGHISTYRPHEPROLEUMETALALEUGLYARGALALEUARGPROGLNSERHISSER

SEQ ID NO: 7 - protein, 2055 aa, Rhodopseudomonas palustris
METASPSERILELEUALAPROARGALAASPALAPROARGASNILEASPGLYALALEUARGGLUSERVALGLNGLNALAALAASPTRPLEUVALALAASNGLNLYSPROASPGLYHISTRPVALGLYARGALAGLUTHRASNALATHRMETGLUALAGLNTRPCYSLEUALALEUTRPPHELEUGLYLEUGLUASPHISPROLEUARGVALARGLEUGLYARGALALEULEUASPTHRGLNARGPROASPGLYALATRPHISVALPHETYRGLYALAPROASNGLYASPILEASNALATHRVALGLUALATYRALAALALEUARGSERLEUGLYHISARGASPASPGLUGLUPROLEUARGLYSALAARGASPTRPILELEUSERLYSGLYGLYLEUALAASNILEARGVALPHETHRARGTYRTRPLEUALALEUILEGLYGLUTRPPROTRPGLULYSTHRPROASNILELEUPROGLUVALILETRPLEUPROTHRTRPPHEPROPHESERILETYRASNPHEALAGLNTRPALAARGALATHRLEUMETPROILEALAVALLEUSERALAHISARGPROSERARGPROLEUALAPROGLNASPARGLEUASPALALEUPHEPROGLNGLYARGASPSERPHEASNTYRASPLEUPROALAARGLEUGLYALAGLYVALTRPASPVALILEPHEARGLYSILEASPTHRILELEUHISARGLEUGLNASPTRPGLYALAARGARGGLYPROHISGLYILEMETARGARGGLYALAILEASPHISVALLEUGLNTRPILEILEARGHISGLNASPTYRASPGLYSERTRPGLYGLYILEGLNPROPROTRPILETYRGLYLEUMETALALEUHISTHRGLUGLYTYRALAMETTHRHISPROVALMETALALYSALALEUASPALALEUASNGLUPROGLYTRPARGILEASPILEGLYASPALATHRPHEILEGLNALATHRASNSERPROVALTRPASPTHRMETLEUSERLEULEUALAPHEASPASPALAGLYLEUGLYGLUARGTYRPROGLUGLNVALGLUARGALAVALARGTRPVALLEULYSARGGLNVALLEUVALPROGLYASPTRPSERVALLYSLEUPROASPVALLYSPROGLYGLYTRPALAPHEGLUTYRALAASNASNPHETYRPROASPTHRASPASPTHRSERVALALALEUMETALALEUALAPROPHEARGHISASPPROLYSTRPGLNALAGLUGLYILEGLUASPALAILEGLNARGGLYILEASPTRPLEUVALALAMETGLNCYSLYSGLUGLYGLYTRPGLYALAPHEASPLYSASPASNASPLYSLYSILELEUALALYSILEPROPHECYSASPPHEGLYGLUALALEUASPPROPROSERALAASPVALTHRALAHISILEILEGLUALAPHEALALYSVALGLYLEUASPARGASNHISPROSERILEVALARGALALEUASPTYRLEULYSARGGLUGLNGLUPROGLUGLYPROTRPPHEGLYARGTRPGLYVALASNTYRVALTYRGLYTHRGLYALAVALLEUPROALALEUALAALAILEGLYGLUASPMETARGGLNPROTYRILEALAARGALACYSASPTRPLEUILEALAARGGLNGLNALAASNGLYGLYTRPGLYGLUSERCYSVALSERTYRMETASPALALYSGLNALAGLYGLUGLYTHRALATHRALASERGLNTHRALATRPALALEUMETALALEUILEALAALAASPARGPROGLNASPARGASPALAILEGLUARGGLYCYSLEUTYRLEUTHRGLUTHRGLNARGASPGLYTHRTRPGLNGLUVALHISTYRTHRGLYTHRGLYPHEPROGLYTYRGLYVALGLYGLNTHRILELYSLEUASNASPPROLEULEUSERLYSARGLEUMETGLNGLYPROGLULEUSERARGSERPHEMETLEUARGTYRASPLEUTYRARGHISTYRPHEPROMETMETALAILEGLYARGVALLEUARGGLNARGGLYASPARGSERGLYHIS

SEQ ID NO: 8 - protein, 2040 aa, Streptomyces coelicolor
METTHRALATHRTHRASPGLYSERTHRGLYALASERLEUARGPROLEUALAALASERALASERASPTHRASPILETHRILEPROALAALAALAALAGLYVALPROGLUALAALAALAARGALATHRARGARGALATHRASPPHELEULEUALALYSGLNASPALAGLUGLYTRPTRPLYSGLYASPLEUGLUTHRASNVALTHRMETASPALAGLUASPLEULEULEUARGGLNPHELEUGLYILEGLNASPGLUGLUTHRTHRARGALAALAALALEUPHEILEARGGLYGLUGLNARGGLUASPGLYTHRTRPALATHRPHETYRGLYGLYPROGLYGLULEUSERTHRTHRILEGLUALATYRVALALALEUARGLEUALAGLYASPSERPROGLUALAPROHISMETALAARGALAALAGLUTRPILEARGSERARGGLYGLYILEALASERALAARGVALPHETHRARGILETRPLEUALALEUPHEGLYTRPTRPLYSTRPASPASPLEUPROGLULEUPROPROGLULEUILETYRPHEPROTHRTRPVALPROLEUASNILETYRASPPHEGLYCYSTRPALAARGGLNTHRILEVALPROLEUTHRILEVALSERALALYSARGPROVALARGPROALAPROPHEPROLEUASPGLULEUHISTHRASPPROALAARGPROASNPROPROARGPROLEUALAPROVALALASERTRPASPGLYALAPHEGLNARGILEASPLYSALALEUHISALATYRARGLYSVALALAPROARGARGLEUARGARGALAALAMETASNSERALAALAARGTRPILEILEGLUARGGLNGLUASNASPGLYCYSTRPGLYGLYILEGLNPROPROALAVALTYRSERVALILEALALEUTYRLEULEUGLYTYRASPLEUGLUHISPROVALMETARGALAGLYLEUGLUSERLEUASPARGPHEALAVALTRPARGGLUASPGLYALAARGMETILEGLUALACYSGLNSERPROVALTRPASPTHRCYSLEUALATHRILEALALEUALAASPALAGLYVALPROGLUASPHISPROGLNLEUVALLYSALASERASPTRPMETLEUGLYGLUGLNILEVALARGPROGLYASPTRPSERVALLYSARGPROGLYLEUPROPROGLYGLYTRPALAPHEGLUPHEHISASNASPASNTYRPROASPILEASPASPTHRALAGLUVALVALLEUALALEUARGARGVALARGHISHISASPPROGLUARGVALGLULYSALAILEGLYARGGLYVALARGTRPASNLEUGLYMETGLNSERLYSASNGLYALATRPGLYALAPHEASPVALASPASNTHRSERALAPHEPROASNARGLEUPROPHECYSASPPHEGLYGLUVALILEASPPROPROSERALAASPVALTHRALAHISVALVALGLUMETLEUALAVALGLUGLYLEUALAHISASPPROARGTHRARGARGGLYILEGLNTRPLEULEUASPALAGLNGLUTHRASPGLYSERTRPPHEGLYARGTRPGLYVALASNTYRVALTYRGLYTHRGLYSERVALILEPROALALEUTHRALAALAGLYLEUPROTHRSERHISPROALAILEARGARGALAVALARGTRPLEUGLUSERVALGLNASNGLUASPGLYGLYTRPGLYGLUASPLEUARGSERTYRARGTYRVALARGGLUTRPSERGLYARGGLYALASERTHRALASERGLNTHRGLYTRPALALEUMETALALEULEUALAALAGLYGLUARGASPSERLYSALAVALGLUARGGLYVALALATRPLEUALAALATHRGLNARGGLUASPGLYSERTRPASPGLUPROTYRPHETHRGLYTHRGLYPHEPROTRPASPPHESERILEASNTYRASNLEUTYRARGGLNVALPHEPROLEUTHRALALEUGLYARGTYRVALHISGLYGLUPROPHEALALYSLYSPROARGALAALAASPALAPROALAGLUALAALAPROALAGLUVALLYSGLYSER

SEQ ID NO: 9 - protein, 1971 aa, Burkholderia ambifaria
METASNASPLEUTHRGLUMETALATHRLEUSERALAGLYTHRVALPROALAGLYLEUASPALAALAVALALASERALATHRASPALALEULEUALAALAGLNASNALAASPGLYHISTRPVALTYRGLULEUGLUALAASPSERTHRILEPROALAGLUTYRVALLEULEUVALHISTYRLEUGLYGLUTHRPROASNLEUGLULEUGLUGLNLYSILEGLYARGTYRLEUARGARGVALGLNGLNALAASPGLYGLYTRPPROLEUPHETHRASPGLYALAPROASNILESERALASERVALLYSALATYRPHEALALEULYSVALILEGLYASPASPGLUASNALAGLUHISMETGLNARGALAARGARGALAILEGLNALAMETGLYGLYALAGLUMETSERASNVALPHETHRARGILEGLNLEUALALEUTYRGLYALAILEPROTRPARGALAVALPROMETMETPROVALGLUILEMETLEULEUPROGLNTRPPHEPROPHEHISLEUSERLYSVALSERTYRTRPALAARGTHRVALILEVALPROLEULEUVALLEUASNALALYSARGPROILEALALYSASNPROARGGLYVALARGILEASPGLULEUPHEVALASPPROPROVALASNALAGLYLEULEUPROARGGLNGLYHISGLNSERPROGLYTRPPHEALAPHEPHEARGVALVALASPHISALALEUARGALAALAASPGLYLEUPHEPROASNTYRTHRARGGLUARGALAILEARGGLNALAVALSERPHEVALASPGLUARGLEUASNGLYGLUASPGLYLEUGLYALAILETYRPROALAMETALAASNALAVALMETMETTYRASPVALLEUGLYTYRALAGLUASPHISPROASNARGALAILEALAARGLYSSERILEGLULYSLEULEUVALVALGLNGLUASPGLUALATYRCYSGLNPROCYSLEUSERPROVALTRPASPTHRSERLEUALAALAHISALALEULEUGLUTHRGLYASPALAARGALAGLUGLUALAVALILEARGGLYLEUGLUTRPLEUARGPROLEUGLNILELEUASPVALARGGLYASPTRPILESERARGARGPROHISVALARGPROGLYGLYTRPALAPHEGLNTYRALAASNPROHISTYRPROASPVALASPASPTHRALAVALVALALAVALALAMETASPARGVALGLNLYSLEULYSHISASNASPALAPHEARGASPSERILEALAARGALAARGGLUTRPVALVALGLYMETGLNSERSERASPGLYGLYTRPGLYALAPHEGLUPROGLUASNTHRGLNTYRTYRLEUASNASNILEPROPHESERASPHISGLYALALEULEUASPPROPROTHRALAASPVALSERGLYARGCYSLEUSERMETLEUALAGLNLEUGLYGLUTHRPROLEUASNSERGLUPROALAARGARGALALEUASPTYRMETLEULYSGLUGLNGLUPROASPGLYSERTRPTYRGLYARGTRPGLYMETASNTYRVALTYRGLYTHRTRPTHRALALEUCYSALALEUASNALAALAGLYLEUTHRPROASPASPPROARGVALLYSARGGLYALAGLNTRPLEULEUSERILEGLNASNLYSASPGLYGLYTRPGLYGLUASPGLYASPSERTYRLYSLEUASNTYRARGGLYPHEGLUGLNALAPROSERTHRALASERGLNTHRALATRPALALEULEUGLYLEUMETALAALAGLYGLUVALASNASNPROALAVALALAARGGLYVALGLUTYRLEUILEALAGLUGLNLYSGLUHISGLYLEUTRPASPGLUTHRARGPHETHRALATHRGLYPHEPROARGVALPHETYRLEUARGTYRHISGLYTYRARGLYSPHEPHEPROLEUTRPALALEUALAARGTYRARGASNLEULYSARGASNASNALATHRARGVALTHRPHEGLYLEU

SEQ ID NO: 10 - protein, 1860 aa, Bacillus anthracis
METSERASNLEULEULEUTYRGLULYSALAHISGLUGLUILEVALARGARGALATHRALALEUGLNTHRMETGLNTRPGLNASPGLYTHRTRPARGPHECYSPHEGLUGLYALAPROLEUTHRASPCYSHISMETILEPHELEULEULYSLEULEUGLYARGASPLYSGLUILEGLUPROPHEVALGLUARGVALALASERLEUGLNTHRASNGLUGLYTHRTRPLYSLEUHISGLUASPGLUVALGLYGLYASNLEUSERALATHRILEGLNSERTYRALAALALEULEUALASERLYSLYSTYRTHRLYSGLUASPALAASNMETLYSARGALAGLUASNPHEILEGLNGLUARGGLYGLYVALALAARGALAHISPHEMETTHRLYSPHELEULEUALAILEHISGLYGLUTYRGLUTYRPROSERLEUPHEHISLEUPROTHRPROILEMETPHELEUGLNASNASPSERPROPHESERILEPHEGLULEUSERSERSERALAARGILEHISLEUILEPROMETMETLEUCYSLEUASNLYSARGPHEARGVALGLYLYSLYSLEULEUPROASNLEUASNHISILEALAGLYGLYGLYGLYGLUTRPPHEARGGLUASPARGSERPROVALPHEGLNTHRLEULEUSERASPVALLYSGLNILEILESERTYRPROLEUSERLEUHISHISLYSGLYTYRGLUGLUILEGLUARGPHEMETLYSGLUARGILEASPGLUASNGLYTHRLEUTYRSERTYRALATHRALASERPHETYRMETILETYRALALEULEUALALEUGLYHISSERLEUGLNSERSERMETILEGLNLYSALAILEALAGLYILETHRSERTYRILETRPLYSMETGLUARGGLYASNHISLEUGLNASNSERPROSERTHRVALTRPASPTHRALALEULEUSERTYRALALEUGLNGLUALAGLNVALSERLYSASPASNLYSMETILEGLNASNALATHRALATYRLEULEULYSLYSGLNHISTHRLYSLYSALAASPTRPSERVALHISALAPROALALEUTHRPROGLYGLYTRPGLYPHESERASPVALASNTHRTHRILEPROASPILEASPASPTHRTHRALAVALLEUARGALALEUALAARGSERARGGLYASNLYSASNILEASPASNALATRPLYSLYSGLYGLYASNTRPILELYSGLYLEUGLNASNASNASPGLYGLYTRPGLYALAPHEGLULYSGLYVALTHRSERLYSLEULEUALALYSLEUPROILEGLUASNALASERASPMETILETHRASPPROSERTHRPROASPILETHRGLYARGVALLEUGLUPHEPHEGLYTHRTYRALAGLNASNGLULEUPROGLULYSGLNILEGLNARGALAILEASNTRPLEUMETASNVALGLNGLUGLUASNGLYSERTRPTYRGLYLYSTRPGLYILECYSTYRLEUTYRGLYTHRTRPALAVALMETTHRGLYLEUARGSERLEUGLYILEPROSERSERASNPROSERLEUTHRARGALAALASERTRPLEUGLUHISILEGLNHISGLUASPGLYGLYTRPGLYGLUSERCYSHISSERSERVALGLULYSARGPHEVALTHRLEUPROPHESERTHRPROSERGLNTHRALATRPALALEUASPALALEUILESERTYRTYRASPTHRGLUTHRPROALAILEARGLYSGLYVALSERTYRLEULEUSERASNPROTYRVALASNGLUARGTYRPROTHRGLYTHRGLYLEUPROGLYALAPHETYRILEARGTYRHISSERTYRALAHISILETYRPROLEULEUTHRLEUALAHISTYRILELYSLYSTYRARGLYS

SEQ ID NO: 11 - protein, 1893 aa, Alicyclobacillus acidocaldarius
METALAGLUGLNLEUVALGLUALAPROALATYRALAARGTHRLEUASPARGALAVALGLUTYRLEULEUSERCYSGLNLYSASPGLUGLYTYRTRPTRPGLYPROLEULEUSERASNVALTHRMETGLUALAGLUTYRVALLEULEUCYSHISILELEUASPARGVALASPARGASPARGMETGLULYSILEARGARGTYRLEULEUHISGLUGLNARGGLUASPGLYTHRTRPALALEUTYRPROGLYGLYPROPROASPLEUASPTHRTHRILEGLUALATYRVALALALEULYSTYRILEGLYMETSERARGASPGLUGLUPROMETGLNLYSALALEUARGPHEILEGLNSERGLNGLYGLYILEGLUSERSERARGVALPHETHRARGMETTRPLEUALALEUVALGLYGLUTYRPROTRPGLULYSVALPROMETVALPROPROGLUILEMETPHELEUGLYLYSARGMETPROLEUASNILETYRGLUPHEGLYSERTRPALAARGALATHRVALVALALALEUSERILEVALMETSERARGGLNPROVALPHEPROLEUPROGLUARGALAARGVALPROGLULEUTYRGLUTHRASPVALPROPROARGARGARGGLYALALYSGLYGLYGLYGLYTRPILEPHEASPALALEUASPARGALALEUHISGLYTYRGLNLYSLEUSERVALHISPROPHEARGARGALAALAGLUILEARGALALEUASPTRPLEULEUGLUARGGLNALAGLYASPGLYSERTRPGLYGLYILEGLNPROPROTRPPHETYRALALEUILEALALEULYSILELEUASPMETTHRGLNHISPROALAPHEILELYSGLYTRPGLUGLYLEUGLULEUTYRGLYVALGLULEUASPTYRGLYGLYTRPMETPHEGLNALASERILESERPROVALTRPASPTHRGLYLEUALAVALLEUALALEUARGALAALAGLYLEUPROALAASPHISASPARGLEUVALLYSALAGLYGLUTRPLEULEUASPARGGLNILETHRVALPROGLYASPTRPALAVALLYSARGPROASNLEULYSPROGLYGLYPHEALAPHEGLNPHEASPASNVALTYRTYRPROASPVALASPASPTHRALAVALVALVALTRPALALEUASNTHRLEUARGLEUPROASPGLUARGARGARGARGASPALAMETTHRLYSGLYPHEARGTRPILEVALGLYMETGLNSERSERASNGLYGLYTRPGLYALATYRASPVALASPASNTHRSERASPLEUPROASNHISILEPROPHECYSASPPHEGLYGLUVALTHRASPPROPROSERGLUASPVALTHRALAHISVALLEUGLUCYSPHEGLYSERPHEGLYTYRASPASPALATRPLYSVALILEARGARGALAVALGLUTYRLEULYSARGGLUGLNLYSPROASPGLYSERTRPPHEGLYARGTRPGLYVALASNTYRLEUTYRGLYTHRGLYALAVALVALSERALALEULYSALAVALGLYILEASPTHRARGGLUPROTYRILEGLNLYSALALEUASPTRPVALGLUGLNHISGLNASNPROASPGLYGLYTRPGLYGLUASPCYSARGSERTYRGLUASPPROALATYRALAGLYLYSGLYALASERTHRPROSERGLNTHRALATRPALALEUMETALALEUILEALAGLYGLYARGALAGLUSERGLUALAALAARGARGGLYVALGLNTYRLEUVALGLUTHRGLNARGPROASPGLYGLYTRPASPGLUPROTYRTYRTHRGLYTHRGLYPHEPROGLYASPPHETYRLEUGLYTYRTHRMETTYRARGHISVALPHEPROTHRLEUALALEUGLYARGTYRLYSGLNALAILEGLUARGARG

SEQ ID NO: 12 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 13 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATTMPLTILSARRPSRPLLPENRLDGLFPEGRENFDYELPVKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 14 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPQKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 15 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPRKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 16 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPKKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 17 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPKKGEEDLWGRFFRAADKGLHSLQSFPVRRFVPREAAIRHVIEWIIRHQDADGGWGGSQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 18 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPKKGEEDLWGRFFRAADKGLHSLQSFPVRRFVPREAAIRHVIEWIIRHQDADGGWGGAQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 19 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGAQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 20 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFASWARATMMPLTILSARRPSRPLLPENRLDGLFPEGRENFDYELPKKGEEDLWGRFFRAADKGLHSLQSFPVRRFVPREAAIRHVIEWIIRHQDADGGWGGSQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 21 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFASWARATMMPLTILSARRPSRPLLPENRLDGLFPEGRENFDYELPRKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 22 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPQKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASEAA

SEQ ID NO: 23 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPQKGEEDLWGRFFRAADKGLHSLQSFPVRRFVPREAAIRHVIEWIIRHQDADGGWGGTQPPWIWGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 24 - protein, 685 aa, synthetic construct; identical to SEQ ID NO: 18

SEQ ID NO: 25 - protein, 685 aa, synthetic construct
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFASWARATMMPLTILSARRPSRPLLPENRLDGLFPEGRENFDYELPKKGEEDLWGRFFRAADKGLHSLQSFPVRRFVPREAAIRHVIEWIIRHQDADGGWGGTQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 26 - protein, 2058 aa, Gluconobacter morbifer
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGGTTGAAAGCAACGCGTGCATGGAGGCGCAGTGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGGTGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCATCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGACCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 27 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGGTGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 28 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCACTATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGGTGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 29 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGCAGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 30 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGGTGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 31 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGCGGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 32 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGAAGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 33 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGAAGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCTCCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 34 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGAAGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCGCCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 35 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGGTGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCGCCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 36 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGGTGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCAACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 37 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGAAGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 38 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGAGCTGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGAAGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCTCCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 39 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGAGCTGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGCGGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCTCCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 40 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGCAGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGAGGCGGCGTAA

SEQ ID NO: 41 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGCAGAAGGGCGAGGAAGATCTGTGGGGTCGTTTCTTTCGTGCGGCGGACAAAGGTCTGCACAGCCTGCAGAGCTTCCCGGTGCGCCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTGGGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGTTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA

SEQ ID NO: 42 - protein, 2058 aa, synthetic construct
ATGAGCCCGGCGGATATCAGCACCAAGAGCAGCAGCTTCCAGCGTCTGGACAACATGCTGCCGGAAGCTGTGAGCAGCGCGTGCGACTGGCTGATTGATCAGCAAAAACCGGATGGCCACTGGGTGGGTCCGCAGGAAAGCAACGCGTGCATGGAGGCGGAATGGTGCCTGGCGCTGTGGTTTCTGGGTCAAGAAGATCACCCGCTGCGTCCGCGTCTGGCGCAGGCGCTGCTGGAAATGCAACGTGAGGACGGCAGCTGGGGTATCTATGTGGGCGCGGACCACGGTGATATTAACACCACCGTTGAGGCGTATGCGGCGCTGCGTAGCATGGGCTATGCGGCGGACATGCCGATCATGGCGAAGAGCGCGGCGTGGATTCAGCAAAAAGGTGGCCTGCGTAACGTGCGTGTTTTCACCCGTTATTGGCTGGCGCTGATCGGTGAATGGCCGTGGGATAAGACCCCGAACCTGCCGCCGGAGATCATTTGGCTGCCGGACAACTTCATCTTTAGCATTTACAACTTTGCGCAATGGGCGCGTGCGACCATGATGCCGCTGACCATTCTGAGCGCGCGTCGTCCGAGCCGTCCGCTGCTGCCGGAAAACCGTCTGGACGGCCTGTTCCCGGAAGGTCGTGAGAACTTTGACTACGAGCTGCCGCGGAAGGGCGAGGAAGATCTGTGGGGTCGTTTCTTTCGTGCGGCGGACAAAGGTCTGCACAGCCTGCAGAGCTTCCCGGTGCGTCGTTTTGTTCCGCGTGAAGCGGCGATCCGTCACGTTATTGAGTGGATCATTCGTCATCAAGATGCGGATGGTGGCTGGGGTGGCACCCAACCGCCGTGGATTTATGGCCTGATGGCGCTGAGCGTGGAAGGTTACCCGCTGCACCACCCGGTTCTGGCGAAGGCGATGGACGCGCTGAACGATCCGGGTTGGCGTCGTGACAAAGGTGATGCGAGCTGGATTCAGGCGAGCAACAGCCCGGTGTGGGACACCATGCTGGCGGTTCTGGCGCTGCATGATGCGGGTGCGGAGGATCGCTATAGCCCGCAGATGGACAAGGCGATCGGCTGGCTGCTGGATCGTCAAGTGCGTGTTAAGGGTGACTGGAGCATTAAACTGCCGGATACCGAACCGGGTGGCTGGGCGTTCGAGTATGCGAACGACAAATACCCGGATACCGATGATACCGCGGTGGCGCTGATTGCGCTGGCGGGTTGCCGTCACCGTCCGGAATGGCGTGAGCGTGATATCGAGGGCGCGATTAGCCGTGGTGTTAACTGGCTGCTGGCGATGCAGAGCAGCAGCGGTGGTTGGGGTGCGTTCGACAAGGATAACAACCGTAGCATCCTGACCAAAATTCCGTTCTGCGACTTTGGTGAAGCGCTGGACCCGCCGAGCGTGGATGTTACCGCGCATGTGCTGGAGGCGTTCGGTCTGCTGGGTATCAGCCGTAACCACCCGAGCGTTCAGAAAGCGCTGGCGTACATTCGTAGCGAACAAGAGCGTAACGGCGCGTGGTTTGGCCGTTGGGGTGTGAACTACGTTTATGGTACCGGTGCGGTTCTGCCGGCGCTGGCGGCGATCGGTGAAGATATGACCCAGCCGTATATTGTGCGTGCGTGCGACTGGCTGATGAGCGTTCAGCAAGAAAACGGTGGCTGGGGCGAGAGCTGCGCGAGCTACATGGATATCAACGCGGTGGGTCACGGCGTTGCGACCGCGAGCCAAACCGCGTGGGCGCTGATTGGTCTGCTGGCGGCGAAGCGTCCGAAAGATCGTGAGGCGATCGCGCGTGGCTGCCAGTTCCTGATTGAACGTCAAGAGGACGGTAGCTGGACCGAGGAAGAGTATACCGGTACCGGCTTTCCGGGTTACGGCGTGGGTCAGGCGATCAAGCTGGACGATCCGAGCCTGCCGGATCGTCTGCTGCAGGGTGCGGAACTGAGCCGTGCGTTCATGCTGCGTTACGACCTGTATCGTCAGTACTTTCCGGTTATGGCGCTGAGCCGTGCGCGTCGTATGATGAAAGAGGACGCGAGCGCGGCGGCGTAA